(19) United States
(12) Patent Application Publication
(10) Pub. No.: US 2026/0199269 A1
(43) Pub. Date: Jul. 16, 2026

(54) OPHTHALMIC PHARMACEUTICAL COMPOSITIONS AND USES THEREOF

(71) Applicant: Alcon Inc., Fribourg (CH)

(72) Inventors: Anjali B. JOSHI, Cary, NC (US); Charliss F. DENNISTON, Chapel Hill, NC (US); Christopher C. COOK, Efland, NC (US); Edward G. RANDLES, Durham, NC (US)

(21) Appl. No.: 19/451,980

(22) Filed: Jan. 16, 2026

Related U.S. Application Data
(60) Provisional application No. 63/746,102, filed on Jan. 16, 2025.
(60) Provisional application No. 63/925,274, filed on Nov. 25, 2025.

(51) Int. Cl. A61K 31/167; A61K 9/00; A61K 47/02; A61K 47/38; A61K 47/44; A61P 27/02

(52) U.S. Cl. CPC A61K 31/167; A61K 9/0048; A61K 47/02; A61K 47/38; A61K 47/44; A61P 27/02

(57) ABSTRACT

Provided herein are topical ophthalmic pharmaceutical compositions of (1R,2S,5R)-2-isopropyl-N-(4-methoxyphenyl)-5-methylcyclohexane-1-carboxamide (WS-12, acoltremon) in LDPE containers, their methods of manufacture, and uses thereof in ocular therapeutics.

### RELATED APPLICATIONS

This application claims priority of U.S. Provisional Patent Application No. 63/746,102, filed Jan. 16, 2025, and U.S. Provisional Patent Application No. 63/925,274, filed Nov. 25, 2025, the entire content of each of which is incorporated herein by reference.

### BACKGROUND

Problematically, containers, including blow-fill-seal (BFS) single use containers, comprising topical ophthalmics for instillation can suffer from reduction of active agent concentration beyond approved limits (e.g., below 90.0% of the label active concentration) over the time-course of their storage. This drop in concentration, and thereby efficacy of an administered volume of the topical ophthalmic, can be exacerbated particularly when the containers are under patient control, which often can include long periods at ambient temperature, e.g., about 20-30° C., for example when left in a handbag, clutch, shoulder bag, luggage, on a counter, in a vehicle, or the like. To address this drop in concentration, some manufacturers may incur excessive cost of materials by filling the containers with an above-label concentration (e.g., over 100% up to about 110% of the label active concentration) and heat curing the filled containers during the manufacturing process to saturate the plastic with the drug thereby mitigating loss of drug to the plastic during subsequent use by the patient. For example, U.S. Ser. No. 11/850,221B2 describes a heat cure manufacturing step to mitigate the drop in drug concentration.

Dry eye, sometimes referred to as xerophthalmia, is a disease characterized by signs and symptoms including stinging, burning, ocular surface inflammation, persistent dryness of the conjunctiva, or opacity of the cornea. Treatments for dry eye include the use of corticosteroids, which may be effective in early stages of the disease, vitamin A supplements, or pilocarpine. The TRPM8 receptor is involved in the control of tear production and its activation using agonists thereof results in increased tear production. Specifically, cold thermoreceptors innervating the cornea in mammals keep tonic trigger activity at normal corneal temperature and are markedly sensitive to minor thermal variations in the eye surface, such as those resulting from evaporation of the precorneal tear film that occurs in the intervals between blinking and during exposure to dry environments. This marked cold sensitivity is the result of a high expression of TRPM8 channels that critically determine a spontaneous basal activity and an increase in the frequency of triggering in response to cold. Moreover, the removal of TRPM8 channels with genetic techniques halves tear secretion in mice. Partial silencing by corneal heating also reduces tear secretion in humans. In light of the above, TRPM8 is a molecular target for the detection of moisture in cold thermoreceptor nerve fibers innervating the exposed eye surface in land animals.

U.S. Ser. No. 11/850,221B2 (and US20240091179A1) describes WS-12, also known as acoltremon, as an agonist of the TRPM8 calcium channel, and useful in treating xerophthalmia or dry eye. The structure of WS-12 is shown below.

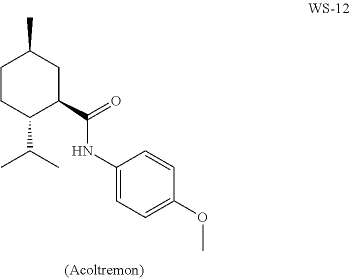 [SMILES: COc1ccc(NC(=O)[C@@H]2C[C@H](C)CC[C@H]2C(C)C)cc1]

It has been found that specific plastic containers, comprising topical ophthalmic acoltremon compositions described herein, obviate these concerns and, importantly, release the patient from cumbersome and compliance-limiting storage restrictions during the lifetime of their possession and use (e.g., about 30 days) of drug product in such plastic containers, and allow manufacturers to reduce additional process steps (e.g., heat curing) and costs of manufacture thereby benefitting the patient and healthcare system on multiple fronts.

### SUMMARY

Provided herein are topical ophthalmic pharmaceutical compositions of (1R,2S,5R)-2-isopropyl-N-(4-methoxyphenyl)-5-methylcyclohexane-1-carboxamide (WS-12, acoltremon) in a low-density polyethylene (LDPE) container and uses thereof. The LDPE-contained ophthalmic pharmaceutical compositions of acoltremon herein address at least 1) a patient's need to keep a given container comprising a topical instillation composition available for use for a period of up to one month or at least about 30 days, 2) a patient's need to conveniently access and store a given container comprising a topical instillation composition at ambient temperature, e.g., about 20-30° C., and 3) the need to provide a consistent concentration and amount of acoltremon in the contained composition over the course of the composition's lifetime from manufacture to use or disposal by the patient.

In some embodiments, provided herein are ophthalmic pharmaceutical compositions, comprising: about 0.0014% w/v to about 0.004% w/v (1R,2S,5R)-2-isopropyl-N-(4-methoxyphenyl)-5-methylcyclohexane-1-carboxamide (acoltremon); about 0.14% w/v hypromellose; about 3.0% w/v polyoxyl 35 castor oil; about 0.78% w/v sodium dihydrogen phosphate dihydrate; about 0.55% w/v NaCl; a sufficient amount of NaOH to provide the ophthalmic pharmaceutical composition with a pH of about 7; and purified water; and wherein the ophthalmic pharmaceutical composition is contained in a LDPE container, the LDPE comprising one or more of a melt mass-flow rate of 0.30 to 0.40 grams per 10 minutes (g/(10-min)), a melting point (aka peak melting point) of about 114° C. (e.g., about 114.0° C.), or a density of about 0.925 g/mL to about 0.927 g/mL.

Also provided herein are methods for the use of the ophthalmic pharmaceutical compositions herein for treating conditions such as dry eye.

Also provided herein are methods of manufacturing the LDPE-contained ophthalmic pharmaceutical compositions herein.

### BRIEF DESCRIPTION OF THE DRAWINGS

FIG. 1 depicts an example of a single-use vial BFS LDPE container, and a connected tear-away strip of 5 single-use vials disposed inside a sealed pouch (e.g., a foil pouch).

FIG. 2 depicts the percent of patients achieving ≥10 mm improvement from baseline in Schirmer score from day 1 to day 90 in patients with dry eye disease as described in Example 3.

FIG. 3 depicts the change from baseline in SANDE score from day 7 to day 90 in patients with dry eye disease as described in Example 3.

FIG. 4 depicts loss of acoltremon in Composition 1 to sorption in DOW 450E LDPE resin BFS single-use vials.

FIG. 5 depicts loss of acoltremon in Composition 1 to sorption in Purell 1840H LDPE resin BFS single-use vials.

FIG. 6 depicts sorption trends of acoltremon to BFS units (made of Sabic PCG00, DOW 20, and Marlex 4538A resins) stored at 30° C. for a period of 4 weeks.

FIG. 7 depicts sorption trends of acoltremon to BFS units (made of Sabic PCG00, DOW 20, and Marlex 4538A resins) stored at 25° C. for a period of 4 weeks.

FIG. 8 depicts sorption trends of acoltremon to BFS units (made of Sabic PCG00 and Purell 3020D LDPE resins) stored at 25° C./60% RH for a period of 32 weeks.

FIG. 9 depicts a trend of acoltremon content loss in pouched BFS units manufactured in Sabic PCG00 LDPE resin with fill volumes of 0.4 ml and stored at 25° C./60% RH for one year.

FIG. 10 depicts trends (normalized) acoltremon content loss in pouched BFS units manufactured in Sabic PCG00 LDPE resin, Purell 3020D LDPE Resin, or Purell 1840H LDPE resin with fill volumes of 0.4 mL or 0.45 mL and stored at 25° C./60% RH for up to 32 weeks.

FIG. 11 depicts the trends of FIG. 10 without normalization.

FIG. 12 depicts trends of acoltremon content loss to LDPE beads in glass vials at 40° C. for 14 days.

FIG. 13 depicts trends (normalized) of acoltremon content loss to polyolefin beads in glass vials at 40° C. for 28 days.

FIG. 14 depicts trends (normalized) of acoltremon content loss in pouched BFS units manufactured in Sabic PCG00 LDPE resin or Purell 1840H LDPE resin with fill volumes of 0.4 mL or 0.45 mL and stored at 25° C./60% RH for up to 1 month.

FIG. 15 depicts trends (normalized) of acoltremon content loss in pouched BFS units manufactured in Sabic PCG00 LDPE resin or Purell 1840H LDPE resin with fill volumes of 0.4 mL or 0.45 mL and stored at 30° C./60% RH for up to 1 month.

FIG. 16 depicts trends (normalized) of acoltremon content loss in pouched BFS units manufactured in Sabic PCG00 LDPE resin or Purell 1840H LDPE resin with fill volumes of 0.4 mL or 0.45 mL and stored at 40° C./60% RH for up to 1 month.

FIG. 17 depicts trends of acoltremon content loss in pouched BFS units manufactured in Purell 3020D LDPE resin or Purell 1840H LDPE resin with fill volumes of 5 mL and stored at 25° C./60% RH or 40° C./60% RH for 2 weeks (40° C.) or 4 weeks (25° C.).

### DETAILED DESCRIPTION

Containers comprising topical ophthalmics for instillation, including BFS packaged eye drops for single use, can suffer from reduction of active agent concentration beyond approved limits (e.g., below 90.0% of the label active concentration) over the time-course of their storage. BFS, is an automated manufacturing process by which plastic containers, such as bottles or ampoules are, in a continuous—typically sterile—operation, blow-formed, filled, and sealed.

U.S. Ser. No. 11/850,221B2 refers to the importance of maintaining “the stability of WS-12 aqueous ophthalmic compositions to maximize the pharmaceutical activity of WS-12 in each dose administered,” and identifies a need for “ophthalmic pharmaceutical compositions comprising WS-12 in a container that can maintain stability of WS-12 with respect to increased temperature, humidity, and exposure to light (and particularly to ultraviolet light), among other types of external effects.” As explained therein, “WS-12, in the ophthalmic composition of the present disclosure, is physically unstable because it interacts with the polyolefin container that it is held in. The sorption processes decrease the effective concentration of WS-12 in the eye drop.”

Furthermore, U.S. Ser. No. 11/850,221B2 identified a trend of increasing percent loss in acoltremon concentration after storage in a polyolefin container for 2 weeks at 40° C. Higher loss of acoltremon to container sorption was directly correlated with decreasing density of LDPE polyolefin and decreasing melting peak temperature of LDPE polyolefin (see Table 12 in U.S. Ser. No. 11/850,221B2).

U.S. Ser. No. 11/850,221B2 addressed these issues via its finding of a process “for stabilizing WS-12-containing compositions disposed within a polymeric (e.g., plastic, e.g., polyolefin) container, which includes exposing the WS-12 composition in its container to a temperature above about 20-25° C. (e.g., at about 40° C.) for at least about 1, 2, 3, 4, 5, 6, 7, 8, 9, or 10 days up to about two weeks.” It was “discovered that this process reduces the variance of WS-12 concentration when subsequently stored at 20-25° C. for about 7, 14, 90, 140, 150, 160, 180, 365, or more days.” Following this method, “WS-12 concentration variance is limited to a decrease of not more than about 6% w/v of WS-12 during storage (e.g., at or below ambient temperature of about 20-25° C.) subsequent to the heat treatment process” (emphasis added).

CEQUA® (cyclosporine ophthalmic solution; a calcineurin inhibitor immunosuppressant indicated to increase tear production in patients with keratoconjunctivitis sicca (dry eye)) prescribing information indicates storage conditions of 20-25° C. EYSUVIS® (loteprednol etabonate ophthalmic suspension; a corticosteroid indicated for the short-term (up to two weeks) treatment of the signs and symptoms of dry eye disease) prescribing information indicates storage conditions of 15-25° C. MIEBO® (perfluorohexyloctane ophthalmic solution; a semifluorinated alkane indicated for treatment of the signs and symptoms of dry eye disease) prescribing information indicates storage conditions of 15-25° C. RESTASIS© (cyclosporine ophthalmic emulsion; a calcineurin inhibitor immunosuppressant indicated to increase tear production in patients whose tear production is presumed to be suppressed due to ocular inflammation associated with keratoconjunctivitis sicca) prescribing information indicates storage conditions of 15-25° C. XIIDRA® (lifitegrast ophthalmic solution; a lymphocyte function-associated antigen-1 (LFA-1) antagonist indicated for the treatment of the signs and symptoms of dry eye disease (DED)) prescribing information indicates storage conditions of 20-25° C. A comparison of storage tolerances is tabulated in Table 1.

TABLE 1
Storage temperature tolerance.

| Temperature | 2° C. | 5° C. | 10° C. | 15° C. | 20° C. | 25° C. | 30° C. |
|---|---|---|---|---|---|---|---|
| CEQUA ® | No | No | No | No | Yes | Yes | No |
| EYSUVIS ® | No | No | No | Yes | Yes | Yes | No |
| MIEBO ® | No | No | No | Yes | Yes | Yes | No |
| RESTASIS ® | No | No | No | Yes | Yes | Yes | No |
| XIIDRA ® | No | No | No | No | Yes | Yes | No |
| Instant Disclosure | Yes | Yes | Yes | Yes | Yes | Yes | Yes |

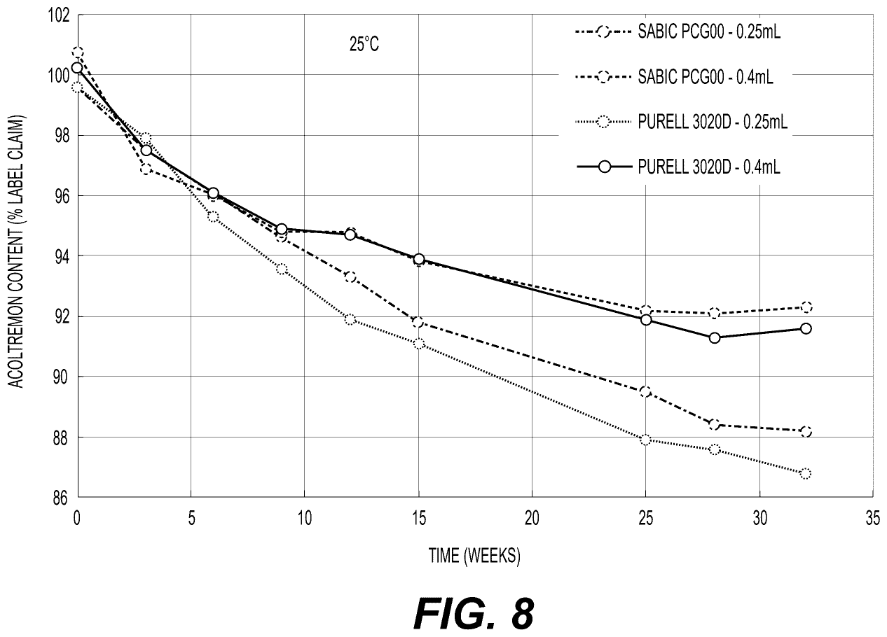

Here, it has been surprisingly found that certain LDPEs (which are suitable for manufacture of blow-fill-seal units), despite having a melt mass-flow rate of 0.30 to 0.40 grams per 10 minutes (g/(10-min)), a density of about 0.925 g/mL to about 0.927 g/mL (e.g., about 0.926 g/mL to about 0.927 g/mL), and/or a melting point of about 114° C. (e.g., about 114.0° C.), do not require a heat curing step as described in U.S. Ser. No. 11/850,221B2 nor refrigeration storage after manufacture, yet can maintain FDA-acceptable label-strength concentrations (±10%) of acoltremon in the topical ophthalmic compositions contained therein for at least 15-30 weeks when stored at temperatures from about 2° C. up to 25° C., or even 30° C., and 60% RH (see FIG. 8 and Table 1). The LDPE containers described herein incur much less (e.g., statistically significant) loss of acoltremon (in a composition herein) to adsorption/absorption than previously described containers. These factors contribute to the improved cost efficacy and ease of manufacturing of the BFS containers and the compositions described herein.

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, the term “about” may explicitly or implicitly modify all numerical values provided herein, unless otherwise indicated.

The term “about” generally indicates a possible variation of a numerical value. In some embodiments, the possible variation (±) may be no more than 10%, 5%, or 1% of the numerical value. In some embodiments, the last decimal place of a numerical value provided herein indicates its degree of accuracy. In some embodiments, where no other error margins are given, the maximum margin is ascertained by applying the rounding-off convention to the last decimal place or last significant digit when a decimal is not present in the given numerical value.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The terms “composition” and “pharmaceutical composition” refer to a mixture of at least one compound described herein with another component, such as a carrier or a pharmaceutically acceptable carrier, respectively.

The term “drug product” refers to a complete form of a medication that is ready to be administered to a patient.

The terms “effective amount” and “therapeutically effective amount” refer to an amount of therapeutic compound, such as a compound described herein, administered to a subject, either as a single dose or as part of a series of doses, which is effective to produce a desired therapeutic effect.

The term “pharmaceutically acceptable carrier” means a pharmaceutically acceptable material, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions or dosage forms described herein are known in the art and described, for example, in “Remington's Pharmaceutical Sciences” (Genaro (Ed.), Mack Publishing Co., 1985), the entire content of which is incorporated herein by reference.

The term “sign” of a disease or disorder is objective evidence of a disease that can be observed or measured.

The term “symptom” of a disease or disorder is the subjective experience of a potential health issue, which cannot be observed by a clinician or anyone other than the person experiencing the symptom.

The term “solid form” includes, but is not limited to, polymorphs, crystalline forms, amorphous forms, solvates, and hydrates of a compound.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order, and may include a combination of one or more embodiments herein, unless otherwise indicated herein or otherwise clearly contradicted by context. The use of examples, or exemplary language (“for example,” “such as,” etc.) provided herein is intended merely to better illuminate the described subject matter and does not pose a limitation on the scope of the subject matter otherwise claimed. No language in the specification should be construed as indicating any non-claimed element essential to practicing the described subject matter.

Furthermore, reference to physical properties, including viscosity and the like, when unqualified by temperature, may be understood, as necessary, in context of the temperature in which the topical ophthalmic compositions here could generally experience following manufacture, e.g., about 2° C. to about 40° C., e.g., about 2° C. to about 25° C.

Each group member of a grouping of alternative elements or embodiments of this disclosure may be referred to and claimed individually or in any combination with other members of the group or other elements found herein. Furthermore, a recited member of a group may be included in, or excluded from, another recited group for reasons of convenience or patentability.

Reference made to a patent or printed publication document throughout this specification incorporates herein by reference the document's entire content.

Embodiments of this disclosure are illustrative. Accordingly, the present disclosure is not limited to that precisely as shown and described.

### Compositions

In some embodiments, provided herein are topical ophthalmic compositions. In some embodiments, the topical ophthalmic compositions herein are provided as drug products or finished dosage forms (e.g., provided in a form in which they will be administered to a patient or subject). Thus, in some embodiments, the topical ophthalmic composition is a drug product. In some embodiments, the drug product is Composition 1 or Composition 2.

In some embodiments, provided herein are LDPE containers having an internal surface, and a topical ophthalmic composition in contact with at least a portion of the internal surface.

Thus, in some embodiments, provided herein are articles of manufacture, comprising a LDPE container having an internal surface, and a topical ophthalmic composition in contact with at least a portion of the internal surface.

It is recognized that the melting point of an LDPE can differ depending on whether the melting point is measured before or after BFS manufacture. That is, commercially supplied LDPE, e.g., supplied as pellets or the like, is provided by the manufacturer with a given set of physical properties, including a melting point. The melting point of the same LDPE after BFS manufacture may change. For consistency, unless otherwise specified, the claims and embodiments herein by default refer to the melting points provided by the commercial supplier of the raw LDPE, but are intended to describe the finished product (e.g., BFS units) LDPE material. Thus, for example, a BFS unit prepared with the LDPE referred to herein as SABIC PCG00, with its physical properties as described herein, may be embodied and claimed as having a melting point of 114° C. (as supplied by the commercial manufacturer) but is understood as equivalent to the melting point, empirically determined by DSC (differential scanning calorimetry) after BFS manufacture, of about 112° C. (see Table 4, below).

The LDPE of the containers or articles of manufacture herein comprises a density of about 0.925 g/mL to about 0.927 g/mL and a melting point of about 114° C. (e.g., about 114.0° C.), optionally also characterized by having a melt mass-flow rate (ISO 1133:2005 or ASTM D1238-10) of about 0.30 to about 0.40 grams per 10 minutes (g/(10-min)), and further optionally characterized by having a heat capacity of about 2.0 J/(g*° C.). In some embodiments, melt mass-flow rate may be abbreviated as MFR. One example of an LDPE herein having a density of about 0.925 g/mL to about 0.926 g/mL, an MFR of 0.33-0.40 g/10 minute, and a melting point of about 114° C. (e.g., about 114.0° C.) is the LDPE commercially sold as “Sabic PCG00,” which has the physical properties described in Table 2. One example of an LDPE herein having a density of about 0.927 g/mL, an MFR of 0.30 g/10 min, a heat capacity of about 2.0 (e.g., 1.976) J/(g*° C.), and a melting point of about 114° C. (e.g., about 114.0° C.) is the LDPE commercially sold as “Purell PE3020D,” which has the physical properties described in Table 3.

TABLE 2
Typical properties of an LDPE commercially sold as “Sabic PCG00.”

| | Metric | | Comments |
|---|---|---|---|
| Physical Properties | | | |
| Density | 0.925 | g/cc | ASTM D1505 |
| Melt Flow | 0.33-0.40 g/10 min | | ISO 1133 |
| | @Load 2.16 kg, | | |
| | Temperature 190° C. | | |
| Mechanical Properties | | | |
| Film Tensile Strength at Yield, TD | 12.0 | MPa | ISO 527-3 |
| Film Elongation at Break, MD | >=200% | | ISO 527-3 |
| Film Elongation at Break, TD | >=500% | | ISO 527-3 |
| Tensile Modulus | 0.220 | GPa | TD; ISO 527-3 |
| Coefficient of Friction | 1 | | ASTM D1894 |
| Tear Strength | 35.0 | kN/m | MD; ISO 6383-2 |
| | 45.0 | kN/m | TD; ISO 6383-2 |
| Dart Drop Total Energy | 250 | J/cm | ASTM D4272 |
| Film Tensile Strength at Break, MD | 28.0 | MPa | ISO 527-3 |
| Film Tensile Strength at Break, TD | 25.0 | MPa | ISO 527-3 |
| Thermal Properties | | | |
| Melting Point | 114° | C. | DIN 53765 |
| Vicat Softening Point | 103° | C. | at 10N (VST/A); ISO 306 |
| Optical Properties | | | |

| | | |
|---|---|---|
| Haze | 8.00% | ASTM D1003 |
| Gloss | 57% | 45°; ASTM D2457 |

TABLE 3
Typical properties of an LDPE commercially
sold as “Purell PE3020D.”

| | Metric | | Comments |
|---|---|---|---|
| Physical Properties | | | |
| Density | 0.927 | g/cc | ASTM D1505 |
| ESCR 100% IGEPAL ® | 16 | hour | ASTM D1693 |
| Melt Flow | 0.30 g/10 min | | ASTM D1238 |
| Mechanical Properties | | | |
| Tensile Strength at Break | 15.2 | MPa | ASTM D638 |
| Tensile Strength, Yield | 15.2 | MPa | ASTM D638 |
| Elongation at Break | 700% | | ASTM D638 |
| Tensile Modulus | 0.434 | GPa | 1% Secant; |
| | | | ASTM D638 |
| Flexural Modulus, 1% Secant | 414 | MPa | ASTM D790 |
| Thermal Properties | | | |
| Deflection Temperature at 0.46 MPa | 46.0° | C. | ASTM D648 |
| (66 psi) | | | |
| Melting Temperature | 114° | C. | ISO 3146 |
| Vicat Softening Point | 102° | C. | ASTM D1525 |

TABLE 4
Typical heat capacity, density, and peak melting
temperature of some commercially available LDPEs.

| | | | | Melt Flow |
|---|---|---|---|---|
| | | | | Rate (g/10- |
| | | Peak Melt | Peak Melt | minute) |
| | | Temperature | Temperature | (@load 2.16 |
| | | (° C.) by DSC | (° C.) | kg, |
| | Density | after BFS | commercially | Temperature |
| LDPE ID | (g/cc) | manufacture | provided | 190° C.) |
| Bormed LE-6600PH | 0.919 | | 110 | 1.5 |
| DOW 450E | 0.923 | | 112 | 2.0 |
| Dow Health 690 | 0.920 | | 112 | 2.0 |
| Dupont 20 | 0.920 | | 108 | 1.9 |
| Dupont 20-6064 | 0.920 | | 109 | 1.9 |
| Purell 1840H (LDPE 5) | 0.919 | 107.8 | 108 | 1.5 |
| Purell 3020D | 0.927 | | 114 | 0.30 |
| Sabic PCG00 | 0.925 | 112.2 | 114 | 0.33-0.4 |

The topical ophthalmic compositions, which can be sterile, of the containers or articles of manufacture herein comprise or consist of:
about 0.0014% w/v to about 0.004% w/v acoltremon (e.g., about 0.0014% w/v to about 0.003% w/v acoltremon or about 0.003% w/v to about 0.004% w/v acoltremon);
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition);
optionally having one or more (e.g., one, two, or three) of: a viscosity of not more than 5 cP (e.g., not more than 4 cP, or even less than 3 cP); an osmolality of about 280 to about 330 mOsm/kg; or a density of about 1.011 g/mL at a temperature of about 20° C. to about 25° C.

Thus, in other embodiments, provided herein are containers or articles of manufacture, comprising an LDPE container having an internal surface, and a topical ophthalmic composition in contact with at least a portion of the internal surface, wherein the LDPE comprises a density of about 0.925 g/mL to about 0.927 g/mL (ASTM D1505) and a melting point of about 114° C. (e.g., about 114.0° C.) (DIN 53765 or ISO 3146), the LDPE optionally further characterized by having a melt mass-flow rate (ISO 1133 or ASTM D1238) of about 0.30 to about 0.33 to about 0.40 g/(10-min) (e.g., about 0.30 g/(10-min) or about 0.33 g/(10-min) to about 0.40 g/(10-min), and the topical ophthalmic composition comprises or consists of:
about 0.0014 to about 0.004% w/v acoltremon (e.g., about 0.0014% w/v to about 0.003% w/v acoltremon or about 0.003% w/v to about 0.004% w/v acoltremon);
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition);
optionally having one or more (e.g., one, two, or three) of: a viscosity of not more than 5 cP (e.g., not more than 4 cP, or even less than 3 cP); an osmolality of about 280 to about 330 mOsm/kg; or a density of about 1.011 g/mL at a temperature of about 20° C. to about 25° C.

In some embodiments, provided herein are containers or articles of manufacture, comprising an LDPE container having an internal surface, and a topical ophthalmic composition in contact with at least a portion of the internal surface, wherein the LDPE comprises a density of about 0.925 g/mL to about 0.926 g/mL (ASTM D1505) and a melt mass-flow rate (ISO 1133 or ASTM D1238) of about 0.33 to about 0.40 g/(10-min), the LDPE optionally further characterized by having a melting point of about 114° C. (e.g., about 114.0° C.) (DIN 53765 or ISO 3146), the melting point being as provided by a commercial supplier of the LDP, or the LDPE optionally further characterized by having a melting point of about 112° C. (e.g., 111.5-112.5° C., e.g., about 112.2° C.) as determined by differential scanning calorimetry (e.g., using about 8-10 mg LDPE sample and a cycle comprising a first heating step of 10° C./min, a quench cooling step, and a second heating step of 10° C./min);
wherein the topical ophthalmic composition comprises or consists of:
about 0.0014 to about 0.004% w/v acoltremon (e.g., about 0.0014% w/v to about 0.003% w/v acoltremon, about 0.003% w/v to about 0.004% w/v acoltremon, about 0.0027% w/v to about 0.0033% w/v, or about 0.003% w/v),
about 0.14% w/v hypromellose,
about 3.0% w/v polyoxyl 35 castor oil,
about 0.78% w/v sodium dihydrogen phosphate dihydrate,
about 0.55% w/v NaCl,
a pH of about 7, and
purified water (e.g., for a fill volume of about 0.25-0.5, about 0.35-0.45, about 0.4, or about 0.25, 0.3, 0.5, 0.6, 0.7, or 1 mL volume of topical ophthalmic composition),
optionally having one or more (e.g., one, two, or three) of: a viscosity of not more than 5 cP (e.g., not more than 4 cP, or even less than 3 cP); an osmolality of about 280 to about 330 mOsm/kg; or a density of about 1.011 g/mL at a temperature of about 20° C. to about 25° C.; and
optionally wherein the topical ophthalmic composition has a volume of about 0.25-0.5, about 0.35-0.45, or about 0.4 mL, or optionally wherein the topical ophthalmic composition has a volume of about 0.25, 0.3, 0.5, 0.6, 0.7, or 1 mL.

In some embodiments, provided herein are containers or articles of manufacture, comprising an LDPE container having an internal surface, and a topical ophthalmic composition in contact with at least a portion of the internal surface, wherein the LDPE comprises a density of about 0.927 g/mL (ASTM D1505) and a melt mass-flow rate (ISO 1133 or ASTM D1238) of about 0.30 g/(10-min), the LDPE optionally further characterized by having a melting point of about 114° C. (e.g., about 114.0° C.) (DIN 53765 or ISO 3146), the melting point being as provided by a commercial supplier of the LDP, or the LDPE optionally further characterized by having a melting point of about 112° C. (e.g., 111.5-112.5° C., e.g., about 112.2° C.) as determined by differential scanning calorimetry (e.g., using about 8-10 mg LDPE sample and a cycle comprising a first heating step of 10° C./min, a quench cooling step, and a second heating step of 10° C./min);
wherein the topical ophthalmic composition comprises or consists of:
about 0.0014 to about 0.004% w/v acoltremon (e.g., about 0.0014% w/v to about 0.003% w/v acoltremon, about 0.003% w/v to about 0.004% w/v acoltremon, about 0.0027% w/v to about 0.0033% w/v, or about 0.003% w/v),
about 0.14% w/v hypromellose,
about 3.0% w/v polyoxyl 35 castor oil,
about 0.78% w/v sodium dihydrogen phosphate dihydrate,
about 0.55% w/v NaCl,
a pH of about 7, and
purified water (e.g., for a fill volume of about 0.25-0.5, about 0.35-0.45, about 0.4, or about 0.25, 0.3, 0.5, 0.6, 0.7, or 1 mL volume of topical ophthalmic composition),
optionally having one or more (e.g., one, two, or three) of: a viscosity of not more than 5 cP (e.g., not more than 4 cP, or even less than 3 cP); an osmolality of about 280 to about 330 mOsm/kg; or a density of about 1.011 g/mL at a temperature of about 20° C. to about 25° C.; and
optionally wherein the topical ophthalmic composition has a volume of about 0.25-0.5, about 0.35-0.45, or about 0.4 mL, or optionally wherein the topical ophthalmic composition has a volume of about 0.25, 0.3, 0.5, 0.6, 0.7, or 1 mL.

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL and a melting point of about 114° C. (e.g., about 114.0° C. (e.g., about 112° C. (e.g., 111.5-112.5° C., e.g., about 112.2° C.) as determined by differential scanning calorimetry (e.g., using about 8-10 mg LDPE sample and a cycle comprising a first heating step of 10° C./min, a quench cooling step, and a second heating step of 10° C./min))), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of: about 0.004% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
about 0.004% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
about 0.004% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
about 0.003% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
about 0.003% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
about 0.003% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
about 0.0014% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
about 0.0014% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
about 0.0014% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.0036 to 0.0044% w/v acoltremon;
0.126 to 0.154% w/v hypromellose;
2.7 to 3.3% w/v polyoxyl 35 castor oil;
0.702 to 0.858% w/v sodium dihydrogen phosphate dihydrate;
0.495 to 0.605% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.0036 to 0.0044% w/v acoltremon;
0.126 to 0.154% w/v hypromellose;
2.7 to 3.3% w/v polyoxyl 35 castor oil;
0.702 to 0.858% w/v sodium dihydrogen phosphate dihydrate;
0.495 to 0.605% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.0036 to 0.0044% w/v acoltremon;
0.126 to 0.154% w/v hypromellose;
2.7 to 3.3% w/v polyoxyl 35 castor oil;
0.702 to 0.858% w/v sodium dihydrogen phosphate dihydrate;
0.495 to 0.605% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.0027 to 0.0033% w/v acoltremon;
0.126 to 0.154% w/v hypromellose;
2.7 to 3.3% w/v polyoxyl 35 castor oil;
0.702 to 0.858% w/v sodium dihydrogen phosphate dihydrate;
0.495 to 0.605% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.0027 to 0.0033% w/v acoltremon;
0.126 to 0.154% w/v hypromellose;
2.7 to 3.3% w/v polyoxyl 35 castor oil;
0.702 to 0.858% w/v sodium dihydrogen phosphate dihydrate;
0.495 to 0.605% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.0027 to 0.0033% w/v acoltremon;
0.126 to 0.154% w/v hypromellose;
2.7 to 3.3% w/v polyoxyl 35 castor oil;
0.702 to 0.858% w/v sodium dihydrogen phosphate dihydrate;
0.495 to 0.605% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.00126 to 0.00154% w/v acoltremon;
0.126 to 0.154% w/v hypromellose;
2.7 to 3.3% w/v polyoxyl 35 castor oil;
0.702 to 0.858% w/v sodium dihydrogen phosphate dihydrate;
0.495 to 0.605% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.00126 to 0.00154% w/v acoltremon;
0.126 to 0.154% w/v hypromellose;
2.7 to 3.3% w/v polyoxyl 35 castor oil;
0.702 to 0.858% w/v sodium dihydrogen phosphate dihydrate;
0.495 to 0.605% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.00126 to 0.00154% w/v acoltremon;
0.126 to 0.154% w/v hypromellose;
2.7 to 3.3% w/v polyoxyl 35 castor oil;
0.702 to 0.858% w/v sodium dihydrogen phosphate dihydrate;
0.495 to 0.605% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.004% w/v acoltremon;
0.14% w/v hypromellose;
3.0% w/v polyoxyl 35 castor oil;
0.78% w/v sodium dihydrogen phosphate dihydrate;
0.55% w/v NaCl;
a pH of 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.004% w/v acoltremon;
0.14% w/v hypromellose;
3.0% w/v polyoxyl 35 castor oil;
0.78% w/v sodium dihydrogen phosphate dihydrate;
0.55% w/v NaCl;
a pH of 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.004% w/v acoltremon;
0.14% w/v hypromellose;
3.0% w/v polyoxyl 35 castor oil;
0.78% w/v sodium dihydrogen phosphate dihydrate;
0.55% w/v NaCl;
a pH of 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.003% w/v acoltremon;
0.14% w/v hypromellose;
3.0% w/v polyoxyl 35 castor oil;
0.78% w/v sodium dihydrogen phosphate dihydrate;
0.55% w/v NaCl;
a pH of 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.003% w/v acoltremon;
0.14% w/v hypromellose;
3.0% w/v polyoxyl 35 castor oil;
0.78% w/v sodium dihydrogen phosphate dihydrate;
0.55% w/v NaCl;
a pH of 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.003% w/v acoltremon;
0.14% w/v hypromellose;
3.0% w/v polyoxyl 35 castor oil;
0.78% w/v sodium dihydrogen phosphate dihydrate;
0.55% w/v NaCl;
a pH of 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.0014% w/v acoltremon;
0.14% w/v hypromellose;
3.0% w/v polyoxyl 35 castor oil;
0.78% w/v sodium dihydrogen phosphate dihydrate;
0.55% w/v NaCl;
a pH of 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.0014% w/v acoltremon;
0.14% w/v hypromellose;
3.0% w/v polyoxyl 35 castor oil;
0.78% w/v sodium dihydrogen phosphate dihydrate;
0.55% w/v NaCl;
a pH of 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and the topical ophthalmic composition comprises or consists of:
0.0014% w/v acoltremon;
0.14% w/v hypromellose;
3.0% w/v polyoxyl 35 castor oil;
0.78% w/v sodium dihydrogen phosphate dihydrate;
0.55% w/v NaCl;
a pH of 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL, the LDPE optionally further characterized by having a melt mass-flow rate (ISO 1133:2005 or ASTM D1238-10) of about 0.30 g/(10-min), and the topical ophthalmic composition comprises or consists of:
about 0.004% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL to about 0.926 g/mL, the LDPE optionally further characterized by having a melt mass-flow rate (ISO 1133:2005 or ASTM D1238-10) of about 0.33 g/(10-min) to about 0.40 g/(10-min), and the topical ophthalmic composition comprises or consists of:
about 0.004% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL, the LDPE optionally further characterized by having a melt mass-flow rate (ISO 1133:2005 or ASTM D1238-10) of about 0.30 g/(10-min), and the topical ophthalmic composition comprises or consists of:
about 0.003% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL to about 0.926 g/mL, the LDPE optionally further characterized by having a melt mass-flow rate (ISO 1133:2005 or ASTM D1238-10) of about 0.33 g/(10-min) to about 0.40 g/(10-min), and the topical ophthalmic composition comprises or consists of:
about 0.003% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.927 g/mL, the LDPE optionally further characterized by having a melt mass-flow rate (ISO 1133:2005 or ASTM D1238-10) of about 0.30 g/(10-min), and the topical ophthalmic composition comprises or consists of:
about 0.0014% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are containers or articles of manufacture, comprising a LDPE container having an internal surface, and a topical ophthalmic composition in contact with at least a portion of the internal surface, wherein the LDPE comprises a density of about 0.925 g/mL to about 0.926 g/mL, the LDPE optionally further characterized by having a melt mass-flow rate (ISO 1133:2005 or ASTM D1238-10) of about 0.33 g/(10-min) to about 0.40 g/(10-min), and the topical ophthalmic composition comprises or consists of:
about 0.0014% w/v acoltremon;
about 0.14% w/v hypromellose;
about 3.0% w/v polyoxyl 35 castor oil;
about 0.78% w/v sodium dihydrogen phosphate dihydrate;
about 0.55% w/v NaCl;
a pH of about 7; and
purified water (e.g., for a fill volume of about 0.25, 0.3, 0.4, 0.5, 0.6, 0.7, or 1 mL volume of topical ophthalmic composition).

In some embodiments, provided herein are topical ophthalmic compositions, wherein the topical ophthalmic composition is contained in a low-density polyethylene (LDPE) container, the LDPE container comprises an internal surface, the topical ophthalmic composition is in contact with at least a portion of the internal surface, the LDPE comprises a density of about 0.925 g/mL to about 0.926 g/mL to about 0.927 g/mL and a melting point of about 114° C., and the topical ophthalmic composition comprises: about 0.0014 to about 0.004% w/v acoltremon (e.g., about 0.0014% w/v to about 0.003% w/v acoltremon or about 0.003% w/v to about 0.004% w/v acoltremon); about 0.14% w/v hypromellose; about 3.0% w/v polyoxyl 35 castor oil; about 0.78% w/v sodium dihydrogen phosphate dihydrate; about 0.55% w/v NaCl; a pH of about 7; and purified water. In some embodiments, the LDPE further comprises a melt mass-flow rate of about 0.30 to about 0.33 to about 0.40 g/(10-min). In some embodiments, the topical ophthalmic compositions further comprise at least one of: a viscosity of not more than 5 cP; an osmolality of about 280 to about 330 mOsm/kg; or a density of about 1.011 g/mL at a temperature of about 20° C. to about 25° C. In some embodiments, the topical ophthalmic compositions have a volume of about 0.25, 0.3, 0.4, 0.5, 0.6, 0.7, or 1 mL. In some embodiments, the topical ophthalmic compositions comprise about 0.0014% w/v acoltremon. In some embodiments, the topical ophthalmic compositions comprise about 0.003% w/v acoltremon. In some embodiments, the topical ophthalmic compositions comprise about 0.004% w/v acoltremon. In some embodiments, the topical ophthalmic compositions comprise: about 0.0014% w/v acoltremon, about 0.003% w/v acoltremon, or about 0.004% w/v acoltremon; about 0.14% w/v hypromellose; about 3.0% w/v polyoxyl 35 castor oil; about 0.78% w/v sodium dihydrogen phosphate dihydrate; about 0.55% w/v NaCl; a pH of about 7; and purified water for a fill volume of about 0.25 mL or about 0.4 mL. In some embodiments, the LDPE container is a blow-fill-seal container or sealed in a foil pouch, or a combination thereof. In some embodiments, the LDPE comprises: a density of about 0.926 g/mL, a melting point of about 114° C., and a melt mass-flow rate of about 0.33 g/10 min; or a density of about 0.927 g/mL, a melting point of about 114° C., and a melt mass-flow rate of about 0.30 g/10 min.

In some embodiments, the LDPE herein is, e.g., has the properties of, an LDPE commercially sold as Sabic PCG00 or Purell PE3020D. In some embodiments, the topical ophthalmic composition within the containers or articles of manufacture herein comprises at least about 0.1 mL of volume. In some embodiments, the topical ophthalmic composition of the containers or articles of manufacture herein comprises at least about 0.25 mL of volume. In some embodiments, the topical ophthalmic composition of the containers or articles of manufacture herein comprises at least about 0.3 mL of volume. In some embodiments, the topical ophthalmic composition of the containers or articles of manufacture herein comprises at least about 0.4 mL of volume. In some embodiments, the topical ophthalmic composition of the containers or articles of manufacture herein comprises at least about 1 mL of volume. In some embodiments, the topical ophthalmic composition of the containers or articles of manufacture herein comprises about 0.1 mL of volume to about 1 L of volume (e.g., about 0.1, 0.2, 0.25, 0.3, 0.4, 0.5, 0.6, 0.7, 1, 5, 10, 50, 100, 200, 500, or 1000 mL). In some embodiments, the articles herein include at least one single use LDPE container.

In some embodiments of the containers or articles of manufacture herein, the article is prepared using blow-fill-seal (or Blow/Fill/Seal or BFS) manufacturing processes. Thus, in some embodiments, the containers or articles herein are BFS-packaged containers or articles. In some embodiments, the articles herein comprise one or more (e.g., 1, 2, 3, 4, 5, 6, 7, 8, 9, or 10) single use BFS-packaged eye drop units. In some embodiments, the LDPE containers herein are BFS units comprising an acoltremon composition described herein. BFS refers to a manufacturing process by which plastic containers, such as bottles or vials, are, in a continuous operation, blow-formed, filled (e.g., with a topical ophthalmic composition, such as those described herein), and sealed. In some embodiments, the BFS units comprise topical ophthalmic composition at a fill volume of about 0.25, 0.3, 0.4, 0.5, 0.6, 0.7, or 1 mL.

In some embodiments, the LDPE is substantially or entirely made from an LDPE having the properties of Sabic PCG00 or Purell PE3020D as described in Table 2, Table 3, or Table 4.

In some embodiments, the topical ophthalmic compositions herein have a liquid volume of about 0.25 mL, which was observed to mitigate drug loss such that the drug concentration (e.g., acoltremon) was not less than 90% of the label claim after about 15 weeks of storage (see FIG. 8). In some embodiments, the topical ophthalmic compositions herein have a liquid volume of about 0.4 mL, which was observed to mitigate drug loss such that the drug concentration (e.g., acoltremon) was not less than 90% of the label claim after about 30 weeks of storage, which is a 100% (2×) improvement in storage duration for only a 60% increase in fill volume when compared with a 0.25 mL fill volume (see FIG. 8).

In some embodiments, the containers or articles of manufacture herein include a packaging material having the LDPE container(s), which contain topical ophthalmics described herein, enclosed therein. In some embodiments, the packaging material includes a pouch, such as a laminate pouch, which may be a foil pouch.

Optionally, the topical ophthalmic compositions described herein can further comprise one or more additional active pharmaceutical ingredients, depending upon the ophthalmic disease or disorder for which the topical ophthalmic composition is to be used to treat or reduce a sign or symptom of a disease or disorder that involves tear production in subject. Examples include, but are not limited to: α1 receptor blockers (including bunazosin or a salt thereof or a solvate thereof such as bunazosin hydrochloride); α2 receptor agonists (including brimonidine or a salt thereof or a solvate thereof such as brimonidine tartrate, and apraclonidine or a salt thereof or a solvate thereof); β-blockers (including carteolol or a salt thereof or a solvate thereof such as carteolol hydrochloride, nipradilol or a salt thereof or a solvate thereof, timolol or a salt thereof or a solvate thereof such as timolol maleate, betaxolol or a salt thereof or a solvate thereof such as betaxolol hydrochloride, levobunolol or a salt thereof or a solvate thereof such as levobunolol hydrochloride, befunolol or a salt thereof or a solvate thereof, atenolol or a salt thereof or a solvate thereof, and metipranolol or a salt thereof or a solvate thereof); carbonic anhydrase inhibitors (including dorzolamide or a salt thereof or a solvate thereof such as dorzolamide hydrochloride, brinzolamide or a salt thereof or a solvate thereof, acetazolamide or a salt thereof or a solvate thereof, dichlorphenamide or a salt thereof or a solvate thereof, and methazolamide or a salt thereof or a solvate thereof); prostaglandins (including their analogs and derivatives (e.g., prostaglandin F2α derivatives) including isopropyl unoprostone or a solvate thereof, tafluprost or a solvate thereof, travoprost or a solvate thereof, bimatoprost or a solvate thereof, latanoprost or a solvate thereof, cloprostenol or a solvate thereof, and fluprostenol or a solvate thereof); Rho kinase inhibitors (including Netarsudil, Ripasudil or a salt thereof or a solvate thereof, Y-39983, and H-1129); sympathomimetic drugs (including dipivefrine or a salt thereof or a solvate thereof such as dipivefrin hydrochloride, and epinephrine or a salt thereof or a solvate thereof such as epinephrine, epinephrine borate, or epinephrine hydrochloride); parasympathomimetic drugs (including distigmine bromide or a salt thereof or a solvate thereof, pilocarpine or a salt thereof or a solvate thereof such as pilocarpine, pilocarpine hydrochloride or pilocarpine nitrate, and carbachol or a salt thereof or a solvate thereof); calcium antagonists (including lomerizine or a salt thereof or a solvate thereof such as lomerizine hydrochloride); and cholinesterase inhibitors (including demecarium or a salt thereof or a solvate thereof, echothiophate or a salt thereof or a solvate thereof, and physostigmine or a salt thereof or a solvate thereof). These APIs can be mixed with acoltremon in the topical ophthalmic composition singly or as combinations of two or more.

In some embodiment, the topical compositions, containers, or articles herein can be stored in a package that blocks transmittance of light, and in particular light having a wavelength ranging from about 1 nm to about 340 nm, such as ultraviolet light. Such a package may include a primary package, such as a container, that is opaque or transparent with respect to visible light. In some embodiments, the primary or a secondary package can comprise at least one substance that blocks transmittance of ultraviolet light, such as a foil. Examples of such packages as well as UV light blocking substances having applications in such packages are disclosed in WO2021195256A1, which is hereby incorporated by reference in its entirety. In some embodiments, an article provided herein may be prepared and placed in a primary or a secondary package or container for storage at a variety of temperatures. Without wishing to be bound by theory, a primary or a secondary package or container useful herein may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some primary or secondary packaging or containers also include the capacity to reduce exposure of their contents to infrared light. Non-limiting examples of a primary or a secondary package or container material that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper, cardboard, paperboard, metallic film, or foil container, or a combination thereof, to further reduce exposure of the container's contents to UV, visible, or infrared light. Compounds and compositions benefiting from reduced discoloration, decomposition, or both during storage, include the acoltremon compositions provided herein. Articles provided herein may need storage lasting up to, or longer than, three months; in some cases, up to, or longer than, one year.

### Methods of Manufacture

The topical ophthalmic compositions described herein may be prepared according to standard liquid solution pharmaceutical manufacturing procedures or according to the protocols described in U.S. Ser. No. 11/850,221B2, which are incorporated herein by reference. The components of the compositions herein are commercially available.

The containers or articles herein (e.g., those containing the topical ophthalmic compositions herein) may be prepared, in some embodiments, according to a method that includes BFS technology to generate an article comprising an LDPE container having an internal surface, and a topical ophthalmic composition in contact with at least a portion of the internal surface. A blow step may be included to form at least a portion of the LDPE container. A fill step may be included to fill the portion of the LDPE container with a topical ophthalmic solution herein. A seal step may be included to form or add a sealed or removable cap part of the LDPE container. The methods of preparation do not include a heat curing step of the article yet the containers or articles are suitable for storage at temperatures convenient to the patient (e.g., up to and including ambient temperature, e.g., up to about 25° C. or up to about 30° C.) for a period that can include all or at least a portion of the time from container or article manufacture through patient acquisition and their subsequent usage or disposal of the article. Thus, in some embodiments the containers or articles can be stably stored at a variety of temperatures, including a range from above freezing (e.g., about 0° C., e.g., about 2° C.) up to about 30° C. In some embodiments the containers or articles are stored at a temperature range from about 2° C. to about 25° C. In some embodiments, the containers or articles herein are not subjected to temperatures above about 30° C. at any time between manufacture, e.g., BFS manufacture, and use or disposal by the patient. Such storage results in maintenance of the label concentration of acoltremon on the container or article within specified tolerances (e.g., FDA or EMA approved tolerances, or not more than 10% variance, e.g., not more than about 6% variance, from the label concentration).

In some embodiments, the methods of manufacture herein include methods for manufacture of a topical ophthalmic composition herein, wherein the LDPE container is prepared by a blow-fill-seal process including a blow step to shape at least a portion of the LDPE container and a fill step to fill the portion of the LDPE container with a volume of the topical ophthalmic composition, and the LDPE container is maintained at a temperature of not more than 30° C. for at least 105, 180, or 210 consecutive days immediately subsequent to the seal step of the blow-fill-seal process.

In some embodiments, the containers or articles herein can be stored at 25° C. for at least 15 weeks without resulting in a drop in acoltremon concentration in the composition disposed therein of more than 10% label claim (e.g., a label claim of 0.003% w/v acoltremon does not decrease beyond 0.0027% w/v acoltremon), such as when the composition volume is about 0.25 mL or more in the container (e.g., a BFS container of Sabic PCG00 or Purell 3020D resin).

In some embodiments, the containers or articles herein can be stored at 25° C. for at least 30 weeks without resulting in a drop in acoltremon concentration in the composition disposed therein of more than 10% label claim (e.g., a label claim of 0.003% w/v acoltremon does not decrease beyond 0.0027% w/v acoltremon), such as when the composition volume is about 0.4 mL or more in the container (e.g., a BFS container of Sabic PCG00 or Purell 3020D resin).

The LDPE containers or articles herein (e.g., a BFS container of Sabic PCG00 or Purell 3020D resin) have an internal surface which has an internal surface area. In some embodiments, the container may have an internal surface area of between about 100 mm² to about 1000 mm², or about 300 mm² to about 800 mm². In some embodiments, the internal surface area is a continuous surface area defining an internal or fillable volume wherein the topical ophthalmic compositions herein are contained. That is to say, the topical ophthalmic compositions herein are filled into the fillable volume of the containers (e.g., during a BFS process). The topical ophthalmic composition comprising acoltremon is in contact with at least a portion of the internal surface of the container.

It has been observed that there is a connection between the internal surface area of the container (e.g., a BFS container of Sabic PCG00 or Purell 3020D resin) and the volume of composition disposed therein. In an embodiment, when a container having the same internal surface area is used, the concentration of acoltremon over time in a container holding 0.4 mL of the composition is higher than the concentration in a container holding 0.25 mL of the same composition. This relationship is demonstrated in FIG. 8, and is another valuable consideration when preparing acoltremon composition BFS articles.

In some embodiments herein, the LDPE container or single-dose vial comprises an internal or fillable volume, and a ratio of internal or fillable volume to filled or liquid volume of the topical ophthalmic composition herein (e.g., Composition 1 or Composition 2) of about 2.2-2.9, about 2.3-2.8, about 2.4-2.7, about 2.4-2.6, about 2.5, or about 1:0.4.

In some embodiments, the LDPE containers or articles herein do not require a heat curing step as described in U.S. Ser. No. 11/850,221B2 nor refrigeration storage after manufacture, yet can maintain FDA-acceptable label-strength concentrations (±10%) of acoltremon in the topical ophthalmic compositions contained therein for at least 15-30 weeks when stored at temperatures up to 25° C. and 60% RH (vide infra). The LDPE containers prepared from Sabic PCG00 or Purell 3020D resin described herein have been found to absorb much less acoltremon than alternative LDPE containers. It is not necessary to fill the presently described containers with an above-label concentration (e.g., over 100% up to about 110% of the label active concentration) to mitigate loss of drug to the polyolefin during storage and subsequent use by the patient. These factors contribute to the cost efficiency, ease of manufacturing of the BFS containers, and the compositions described herein.

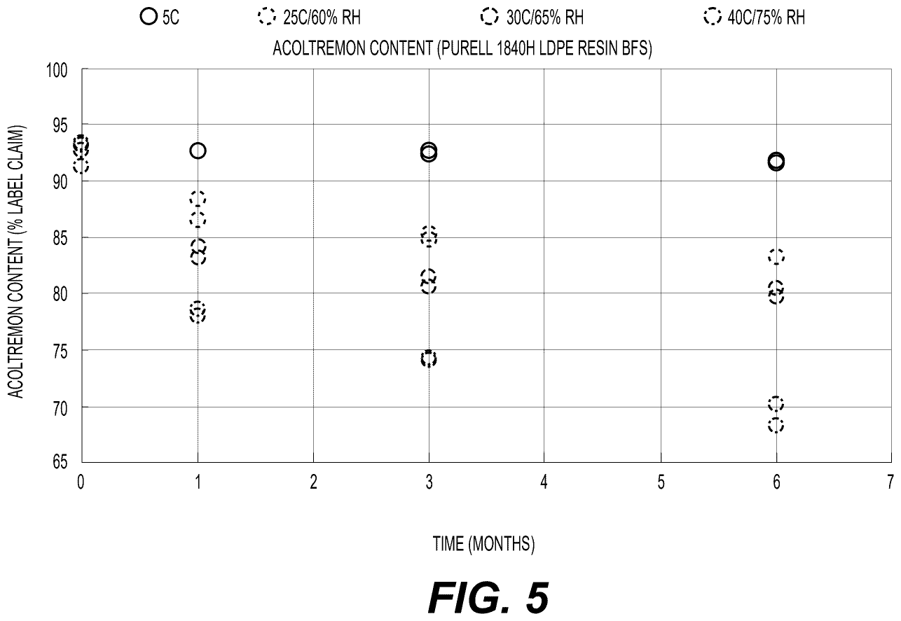

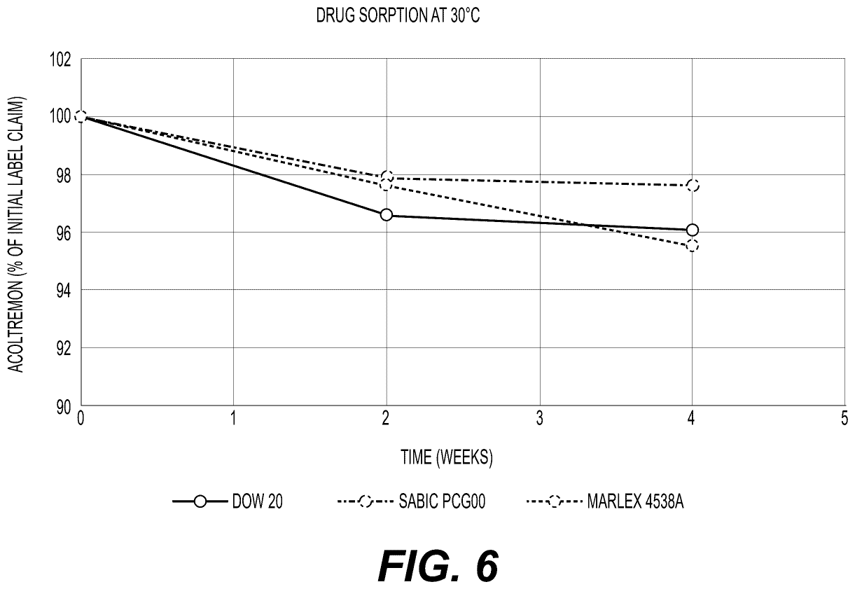

In some embodiments, BFS containers having the properties of the Sabic PCG00 or Purell 3020D resin herein absorb about 1% to about 90% less acoltremon than other polyolefin containers tested. In some embodiments, a Sabic PCG00 container absorbed about 2% less acoltremon than a DOW 20 container and a Marlex 4538A container after 4 weeks at 30° C. and 25° C. (see FIG. 5 and FIG. 6, respectively).

In some embodiments, provided herein are methods of manufacturing a single-dose vial, the single-dose vial comprising a liquid volume (e.g., about 0.4 mL) of a topical ophthalmic composition herein (e.g., Composition 1 or Composition 2) filled in the single-dose vial,
wherein the single-dose vial is prepared by a blow-fill-seal (BFS) process, the BFS process comprising blow-forming a low-density polyethylene (LDPE) resin to form a blown vial, filling the blown vial with the liquid volume to form a blown and filled vial, and sealing the blown and filled vial to form the single-dose vial, and
wherein the LDPE resin comprises at least two (e.g., all three) of a melt mass-flow rate of 0.30 to 0.40 grams per 10 minutes (g/(10-min)) (e.g., 0.33 to 0.40 g/(10-min)), a density of 0.925 g/mL to 0.927 g/mL (e.g., 0.925 g/mL), or a peak melting point of 114° C.,
optionally wherein the method further comprises, less than one hour after the BFS process, maintaining the single-dose vial at a temperature of not less than about 2° C. and not more than about 30° C. (e.g., not more than about 25° C., e.g., about 20-25° C., e.g., not less than 10° C. or not less than 15° C., or a combination thereof) for a time period of at least 15 weeks (e.g., at least 26 weeks or at least 30 weeks), up to 32 weeks, or up to a year, or longer, or a combination thereof.

In some embodiments, provided herein are methods of manufacturing a single-dose vial, the single-dose vial comprising a liquid volume (e.g., about 0.4 mL) of a topical ophthalmic composition herein (e.g., Composition 1 or Composition 2) filled in the single-dose vial,
wherein the single-dose vial is prepared by a blow-fill-seal (BFS) process, the BFS process comprising blow-forming a low-density polyethylene (LDPE) resin to form a blown vial, filling the blown vial with the liquid volume to form a blown and filled vial, and sealing the blown and filled vial to form the single-dose vial, and
wherein the LDPE resin comprises a melt mass-flow rate of 0.33 to 0.40 g/(10-min), a density of 0.925 g/mL, and a peak melting point of 114° C.,
optionally wherein the method further comprises, less than one hour after the BFS process, maintaining the single-dose vial at a temperature of not less than about 2° C. and not more than about 30° C. (e.g., not more than about 25° C., e.g., about 20-25° C., e.g., not less than 10° C. or not less than 15° C., or a combination thereof) for a time period of at least 15 weeks (e.g., at least 26 weeks or at least 30 weeks), up to 32 weeks, or up to a year, or longer, or a combination thereof.

In some embodiments of the methods of manufacturing herein, the LDPE resin is Sabic PCG00 or an LDPE resin having the physical properties (e.g., two or more of density, MFR, or melting point) about (e.g., ±10%) of those of Sabic PCG00 described herein.

In some embodiments, provided herein are methods of manufacturing a single-dose vial, the single-dose vial comprising a liquid volume (e.g., about 0.4 mL) of a topical ophthalmic composition herein (e.g., Composition 1 or Composition 2) filled in the single-dose vial,
wherein the single-dose vial is prepared by a blow-fill-seal (BFS) process, the BFS process comprising blow-forming a low-density polyethylene (LDPE) resin to form a blown vial, filling the blown vial with the liquid volume to form a blown and filled vial, and sealing the blown and filled vial to form the single-dose vial, and
wherein the LDPE resin comprises a melt mass-flow rate of 0.30 g/(10-min), a density of 0.927 g/mL, and a peak melting point of 114° C.,
optionally wherein the method further comprises, less than one hour after the BFS process, maintaining the single-dose vial at a temperature of not less than about 2° C. and not more than about 30° C. (e.g., not more than about 25° C., e.g., about 20-25° C., e.g., not less than 10° C. or not less than 15° C., or a combination thereof) for a time period of at least 15 weeks (e.g., at least 26 weeks or at least 30 weeks), up to 32 weeks, or up to a year, or longer, or a combination thereof.

In some embodiments of the methods of manufacturing herein, the LDPE resin is Purell 3020D or an LDPE resin having the physical properties (e.g., two or more of density, MFR, or melting point) about (e.g., ±10%) of those of Purell 3020D described herein.

The resin MFRs, densities, and melting temperatures may be as provided by the commercial manufacturer. The resin MFRs herein may be determined by ASTM D1238. The resin densities herein may be determined by ASTM D1505. The resin melting temperatures herein may be determined by ISO 3146, or by differential scanning calorimetry (DSC) as described in Example 9.

### Methods of Use

The topical ophthalmic compositions described herein disposed within the LDPE containers described herein are useful in treating dry eye or its signs or symptoms in subjects in need thereof (e.g., patients). For example, such uses are described in U.S. Ser. No. 11/850,221B2 and incorporated herein by reference.

Thus, in some embodiments, articles herein are useful in methods of treating or reducing ocular irritation involving tear production in a subject in need thereof, or treating or reducing at least one sign or symptom of an ophthalmic disease or disorder involving tear production in a subject in need thereof.

In some embodiments, the articles herein are useful in various methods for treating, reducing, ameliorating, reducing the likelihood of, or preventing an ophthalmic disease or disorder, a sign or symptom of such a disease or disorder, or a combination thereof, in a subject in need of such a method. In a particular embodiment, the disease or disorder involves tear production, such as dry eye (xerophthalmia), keratoconjunctivitis sicca, or Sjögren's syndrome. Thus, the present disclosure extends to a method for treating or reducing the likelihood of dry eye in a subject, comprising administering a therapeutically effective amount of a topical ophthalmic composition from the articles of the present disclosure.

Applicable ophthalmic diseases or disorders for which the topical ophthalmic composition contained within the articles herein include but are not limited to dry eye or Sjögren's syndrome. The term “dry eye” is understood according to the definition of dry eye provided by TFOS DEWS II (summarized in Craig et al, The Ocular Surface 2017, 15, 276-283) (“Dry eye is a multifactorial disease of the ocular surface characterized by a loss of homeostasis of the tear film, and accompanied by ocular symptoms, in which tear film instability and hyperosmolarity, ocular surface inflammation and damage, and neurosensory abnormalities play etiological roles”). Dry eye occurs when either the eye does not produce enough tears or when the tears evaporate too quickly. Multiple causes can lead to dry eye, which is more common in elderly people. Causes of dry eye include but are not limited to: meibomian gland dysfunction (MGD) and other ocular inflammatory processes, blink-related causes, ocular irritations such as contact lens-related causes, vitamin A deficiency, Sjögren's syndrome, rheumatoid arthritis and other rheumatological disease and disorders, chemical or thermal burns, drugs such as atenolol, chlorpheniramine, diuretics, hydrochlorothiazide, isotretinoin, ketorolac, ketotifen, levocabastin, levofloxacin, oxybutynin, and tolterodine. Sjögren's syndrome is an immune disorder characterized by a dry mouth and/or dry eyes.

In some embodiments, provided herein are methods of treating an ophthalmic disease or condition in a subject in need thereof, the disease or condition involving tear production and/or reducing symptoms thereof, the methods comprising topically administering (e.g., via instillation to an eye of the subject) the topical ophthalmic composition contained in the articles herein (e.g, administered from a single-dose vial as manufactured herein) to a subject in need thereof.

In some embodiments, provided herein are methods of treating an ophthalmic disease or condition in a subject in need thereof, the disease or condition involving tear production and/or reducing symptoms thereof, the methods comprising topically administering (e.g., via instillation to an eye of the subject) an administered volume of a topical ophthalmic composition herein (e.g., Composition 1 or Composition 2) from a single-dose vial to a subject in need thereof,
wherein the single-dose vial is prepared by a method of manufacturing the single-dose vial, the single-dose vial comprising a liquid volume (e.g., about 0.4 mL) of the topical ophthalmic composition herein (e.g., Composition 1 or Composition 2) filled in the single-dose vial,
wherein the method of manufacturing the single-dose vial includes a blow-fill-seal (BFS) process, the BFS process comprising blow-forming a low-density polyethylene (LDPE) resin to form a blown vial, filling the blown vial with the liquid volume to form a blown and filled vial, and sealing the blown and filled vial to form the single-dose vial, and
wherein the LDPE resin comprises at least two (e.g., all three) of a melt mass-flow rate of 0.30 to 0.40 grams per 10 minutes (g/(10-min)) (e.g., 0.33 to 0.40 g/(10-min)), a density of 0.925 g/mL to 0.927 g/mL (e.g., 0.925 g/mL), or a peak melting point of 114° C.,
optionally wherein the method further comprises, less than one hour after the BFS process, maintaining the single-dose vial at a temperature of not less than about 2° C. and not more than about 30° C. (e.g., not more than about 25° C., e.g., about 20-25° C., e.g., not less than 10° C. or not less than 15° C., or a combination thereof) for a time period of at least 15 weeks (e.g., at least 26 weeks or at least 30 weeks), up to 32 weeks, or up to a year, or longer, or a combination thereof, and
optionally wherein the administered volume is about 25 to about 40 μL.

In some embodiments, provided herein are methods of treating an ophthalmic disease or condition in a subject in need thereof, the disease or condition involving tear production and/or reducing symptoms thereof, the methods comprising topically administering (e.g., via instillation to an eye of the subject) an administered volume of a topical ophthalmic composition herein (e.g., Composition 1 or Composition 2) from a single-dose vial to a subject in need thereof,
wherein the single-dose vial is prepared by a method of manufacturing the single-dose vial, the single-dose vial comprising a liquid volume (e.g., about 0.4 mL) of the topical ophthalmic composition herein (e.g., Composition 1 or Composition 2) filled in the single-dose vial,
wherein the method of manufacturing the single-dose vial includes a blow-fill-seal (BFS) process, the BFS process comprising blow-forming a low-density polyethylene (LDPE) resin to form a blown vial, filling the blown vial with the liquid volume to form a blown and filled vial, and sealing the blown and filled vial to form the single-dose vial, and
wherein the LDPE resin comprises a melt mass-flow rate of 0.33 to 0.40 g/(10-min), a density of 0.925 g/mL, and a peak melting point of 114° C.,
optionally wherein the method further comprises, less than one hour after the BFS process, maintaining the single-dose vial at a temperature of not less than about 2° C. and not more than about 30° C. (e.g., not more than about 25° C., e.g., about 20-25° C., e.g., not less than 10° C. or not less than 15° C., or a combination thereof) for a time period of at least 15 weeks (e.g., at least 26 weeks or at least 30 weeks), up to 32 weeks, or up to a year, or longer, or a combination thereof, and
optionally wherein the administered volume is about 25 to about 40 μL.

In some embodiments of the methods of treating herein, the LDPE resin is Sabic PCG00 or an LDPE resin having the physical properties (e.g., two or more of density, MFR, or melting point) about (e.g., ±10%) of those of Sabic PCG00 described herein.

In some embodiments, provided herein are methods of treating an ophthalmic disease or condition in a subject in need thereof, the disease or condition involving tear production and/or reducing symptoms thereof, the methods comprising topically administering (e.g., via instillation to an eye of the subject) an administered volume of a topical ophthalmic composition herein (e.g., Composition 1 or Composition 2) from a single-dose vial to a subject in need thereof,
wherein the single-dose vial is prepared by a method of manufacturing the single-dose vial, the single-dose vial comprising a liquid volume (e.g., about 0.4 mL) of the topical ophthalmic composition herein (e.g., Composition 1 or Composition 2) filled in the single-dose vial,
wherein the method of manufacturing the single-dose vial includes a blow-fill-seal (BFS) process, the BFS process comprising blow-forming a low-density polyethylene (LDPE) resin to form a blown vial, filling the blown vial with the liquid volume to form a blown and filled vial, and sealing the blown and filled vial to form the single-dose vial, and
wherein the LDPE resin comprises a melt mass-flow rate of 0.30 g/(10-min), a density of 0.927 g/mL, and a peak melting point of 114° C.,
optionally wherein the method further comprises, less than one hour after the BFS process, maintaining the single-dose vial at a temperature of not less than about 2° C. and not more than about 30° C. (e.g., not more than about 25° C., e.g., about 20-25° C., e.g., not less than 10° C. or not less than 15° C., or a combination thereof) for a time period of at least 15 weeks (e.g., at least 26 weeks or at least 30 weeks), up to 32 weeks, or up to a year, or longer, or a combination thereof, and
optionally wherein the administered volume is about 25 to about 40 μL.

In some embodiments of the methods of treating herein, the LDPE resin is Purell 3020D or an LDPE resin having the physical properties (e.g., two or more of density, MFR, or melting point) about (e.g., ±10%) of those of Purell 3020D described herein.

In some embodiments, provided herein are methods of treating an ophthalmic disease or condition in a subject in need thereof, the disease or condition involving tear production and/or reducing symptoms thereof, the methods comprising topically administering (e.g., via instillation to an eye of the subject) the topical ophthalmic composition contained in the articles herein to a subject in need thereof,
wherein binding of the acoltremon in the topical ophthalmic composition to a TRPM8 receptor modulates the activity of the TRPM8 channel,
wherein the binding of the molecule to the TRPM8 receptor increases or decreases tear secretion caused by ophthalmic disease or condition, and
wherein the concentration of the molecule needed to bind and modulate the TRPM8 receptor in tissues is not sufficient to be cytotoxic to the subject in need thereof.

In some embodiments of the methods herein, the methods stop, reduce, or prophylactically prevent the likelihood of an unwanted physiological change or disorder (e.g., certain clinical outcomes), such as dryness of the eyes. Such clinical outcomes include, without limitation, observational symptom relief, reduction of disease extent, stabilized pathological condition (specifically not worsened), delayed or stopped disease progression, improved or palliated pathological condition and remission (both partial and total). Subjects needing treatment include subjects already suffering from the disease or disorder, as well as those susceptible of suffering from the disease or disorder or those for whom the disease or disorder should be prevented, e.g., are predisposed to acquiring the disease or disorder.

In some embodiments, provided herein are methods of treating one or more signs or symptoms of dry eye disease in a subject in need thereof, comprising administration to the subject of a topical ophthalmic composition from an LDPE container having an internal surface, wherein the LDPE comprises a density of about 0.925 g/mL to about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   about 0.0014% w/v to about 0.004% w/v acoltremon (e.g., about 0.0014% w/v to about 0.003% w/v acoltremon or about 0.003% w/v to about 0.004% w/v acoltremon);
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface, wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   about 0.004% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface, wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   about 0.004% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   about 0.004% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   about 0.003% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   about 0.003% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   about 0.003% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   about 0.0014% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   about 0.0014% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   about 0.0014% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises: 0.0034 to 0.0046% w/v acoltremon;
   0.119 to 0.161% w/v hypromellose;
   2.55 to 3.45% w/v polyoxyl 35 castor oil;
   0.663 to 0.897% w/v sodium dihydrogen phosphate dihydrate;
   0.4675 to 0.6325% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises: 0.0034 to 0.0046% w/v acoltremon;
   0.119 to 0.161% w/v hypromellose;
   2.55 to 3.45% w/v polyoxyl 35 castor oil;
   0.663 to 0.897% w/v sodium dihydrogen phosphate dihydrate;
   0.4675 to 0.6325% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises: 0.0034 to 0.0046% w/v acoltremon;
   0.119 to 0.161% w/v hypromellose;
   2.55 to 3.45% w/v polyoxyl 35 castor oil;
   0.663 to 0.897% w/v sodium dihydrogen phosphate dihydrate;
   0.4675 to 0.6325% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises: 0.00255 to 0.00345% w/v acoltremon;
   0.119 to 0.161% w/v hypromellose;
   2.55 to 3.45% w/v polyoxyl 35 castor oil;
   0.663 to 0.897% w/v sodium dihydrogen phosphate dihydrate;
   0.4675 to 0.6325% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises: 0.00255 to 0.00345% w/v acoltremon;
   0.119 to 0.161% w/v hypromellose;
   2.55 to 3.45% w/v polyoxyl 35 castor oil;
   0.663 to 0.897% w/v sodium dihydrogen phosphate dihydrate;
   0.4675 to 0.6325% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.00255 to 0.00345% w/v acoltremon;
   0.119 to 0.161% w/v hypromellose;
   2.55 to 3.45% w/v polyoxyl 35 castor oil;
   0.663 to 0.897% w/v sodium dihydrogen phosphate dihydrate;
   0.4675 to 0.6325% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.00119 to 0.00161% w/v acoltremon;
   0.119 to 0.161% w/v hypromellose;
   2.55 to 3.45% w/v polyoxyl 35 castor oil;
   0.663 to 0.897% w/v sodium dihydrogen phosphate dihydrate;
   0.4675 to 0.6325% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.00119 to 0.00161% w/v acoltremon;
   0.119 to 0.161% w/v hypromellose;
   2.55 to 3.45% w/v polyoxyl 35 castor oil;
   0.663 to 0.897% w/v sodium dihydrogen phosphate dihydrate;
   0.4675 to 0.6325% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.00119 to 0.00161% w/v acoltremon;
   0.119 to 0.161% w/v hypromellose;
   2.55 to 3.45% w/v polyoxyl 35 castor oil;
   0.663 to 0.897% w/v sodium dihydrogen phosphate dihydrate;
   0.4675 to 0.6325% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.004% w/v acoltremon;
   0.14% w/v hypromellose;
   3.0% w/v polyoxyl 35 castor oil;
   0.78% w/v sodium dihydrogen phosphate dihydrate;
   0.55% w/v NaCl;
   a pH of 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.004% w/v acoltremon;
   0.14% w/v hypromellose;
   3.0% w/v polyoxyl 35 castor oil;
   0.78% w/v sodium dihydrogen phosphate dihydrate;
   0.55% w/v NaCl;
   a pH of 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.004% w/v acoltremon;
   0.14% w/v hypromellose;
   3.0% w/v polyoxyl 35 castor oil;
   0.78% w/v sodium dihydrogen phosphate dihydrate;
   0.55% w/v NaCl;
   a pH of 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.003% w/v acoltremon;
   0.14% w/v hypromellose;
   3.0% w/v polyoxyl 35 castor oil;
   0.78% w/v sodium dihydrogen phosphate dihydrate;
   0.55% w/v NaCl;
   a pH of 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.003% w/v acoltremon;
   0.14% w/v hypromellose;
   3.0% w/v polyoxyl 35 castor oil;
   0.78% w/v sodium dihydrogen phosphate dihydrate;
   0.55% w/v NaCl;
   a pH of 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.003% w/v acoltremon;
   0.14% w/v hypromellose;
   3.0% w/v polyoxyl 35 castor oil;
   0.78% w/v sodium dihydrogen phosphate dihydrate;
   0.55% w/v NaCl;
   a pH of 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.0014% w/v acoltremon;
   0.14% w/v hypromellose;
   3.0% w/v polyoxyl 35 castor oil;
   0.78% w/v sodium dihydrogen phosphate dihydrate;
   0.55% w/v NaCl;
   a pH of 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.926 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.0014% w/v acoltremon;
   0.14% w/v hypromellose;
   3.0% w/v polyoxyl 35 castor oil;
   0.78% w/v sodium dihydrogen phosphate dihydrate;
   0.55% w/v NaCl;
   a pH of 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL and a melting point of about 114° C. (e.g., about 114.0° C.), the LDPE optionally further characterized by having a melt mass-flow rate (ISO 1133:2005 or ASTM D1238-10) of about 0.30 to about 0.33 to about 0.40 g/(10-min), and
wherein the topical ophthalmic composition, which can be sterile, is in contact with at least a portion of the internal surface, and comprises:
   0.0014% w/v acoltremon;
   0.14% w/v hypromellose;
   3.0% w/v polyoxyl 35 castor oil;
   0.78% w/v sodium dihydrogen phosphate dihydrate;
   0.55% w/v NaCl;
   a pH of 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL, the LDPE optionally further characterized by having a melt mass-flow rate (ISO 1133:2005 or ASTM D1238-10) of about 0.30 g/(10-min), and
wherein the topical ophthalmic composition, which can be sterile, is in contact with at least a portion of the internal surface, and comprises:
   about 0.004% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL to about 0.926 g/mL, the LDPE optionally further characterized by having a melt mass-flow rate (ISO 1133:2005 or ASTM D1238-10) of about 0.33 g/(10-min) to about 0.4 g/(10-min), and
wherein the topical ophthalmic composition, which can be sterile, is in contact with at least a portion of the internal surface, and comprises:
   about 0.004% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL, the LDPE optionally further characterized by having a melt mass-flow rate (ISO 1133:2005 or ASTM D1238-10) of about 0.30 g/(10-min), and
wherein the topical ophthalmic composition, which can be sterile, is in contact with at least a portion of the internal surface, and comprises:
   about 0.003% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL to about 0.926 g/mL, the LDPE optionally further characterized by having a melt mass-flow rate (ISO 1133:2005 or ASTM D1238-10) of about 0.33 g/(10-min) to about 0.4 g/(10-min), and
wherein the topical ophthalmic composition, which can be sterile, is in contact with at least a portion of the internal surface, and comprises:
   about 0.003% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.927 g/mL, the LDPE optionally further characterized by having a melt mass-flow rate (ISO 1133:2005 or ASTM D1238-10) of about 0.30 g/(10-min), and
wherein the topical ophthalmic composition, which can be sterile, is in contact with at least a portion of the internal surface, and comprises:
   about 0.0014% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments, provided herein are methods of treating one or more signs or symptoms of dry eye disease in a subject in need thereof, comprising administration to the subject of a topical ophthalmic composition from an LDPE container having an internal surface,
wherein the LDPE comprises a density of about 0.925 g/mL to about 0.926 g/mL, the LDPE optionally further characterized by having a melt mass-flow rate (ISO 1133:2005 or ASTM D1238-10) of about 0.33 g/(10-min) to about 0.4 g/(10-min), and
wherein the topical ophthalmic composition, which can be sterile, is in contact with at least a portion of the internal surface, and comprises:
   about 0.0014% w/v acoltremon;
   about 0.14% w/v hypromellose;
   about 3.0% w/v polyoxyl 35 castor oil;
   about 0.78% w/v sodium dihydrogen phosphate dihydrate;
   about 0.55% w/v NaCl;
   a pH of about 7; and
   purified water (e.g., for a fill volume of about 0.25, 0.3, 0.4, 0.5, 0.6, 0.7, or 1 mL volume of topical ophthalmic composition).

In some embodiments of these methods, the LDPE herein is, e.g., has the properties of, an LDPE commercially sold as Sabic PCG00 or Purell PE3020D.

In some embodiments of these methods, the LDPE container or article has not been exposed to a temperature above 30° C. subsequent to its manufacture.

In some embodiments of these methods, the LDPE container or article is maintained between a temperature range from above freezing (e.g., about 0° C.) up to about 30° C. subsequent to its manufacture (e.g., from 2-25° C.).

In some embodiments of the methods herein, the amount of the topical ophthalmic composition administered to the subject is about 0.025 mL to about 0.05 mL, e.g., about 0.025 mL to about 0.03 mL, about 0.03 to about 0.04 mL, or about 0.04 mL to about 0.05 mL, to an eye of the subject. In some embodiments of the methods herein, the amount is administered once or twice daily to an eye (e.g., each eye) of the subject. In some embodiments of the methods herein the amount is administered once or twice daily to an eye (e.g., each eye) of the subject for more than 14 days, e.g., daily for more than 28 consecutive days, e.g., daily for more than 60 consecutive days.

In some embodiments, provided herein are methods of treating one or more signs or symptoms of dry eye disease in a subject in need thereof, comprising instillation of a dosage amount of a topical ophthalmic composition herein to an eye of the subject. In some embodiments, the dosage amount has a volume of about 0.025, 0.03, 0.04, 0.05, 0.06, or 0.07 mL. In some embodiments, the topical ophthalmic composition has not been exposed to a temperature above 30° C. subsequent to its being contained in the LDPE container. In some embodiments, the topical ophthalmic composition is maintained between a temperature range of above freezing to about 30° C. subsequent to its being contained in the LDPE container. In some embodiments, the dosage amount has a volume of about 0.025 mL to about 0.05 mL (e.g., about 0.03 to about 0.04 mL). In some embodiments, the instillation is once or twice daily to each eye of the subject. In some embodiments, the instillation is once or twice daily to each eye of the subject for more than 14 consecutive days. In some embodiments, the instillation is once or twice daily to each eye of the subject for more than 28 consecutive days. In some embodiments, the instillation is once or twice daily to each eye of the subject for more than 60 consecutive days.

In some embodiments, provided herein are methods of treating one or more signs or symptoms of dry eye disease in a subject in need thereof, comprising instillation of a topical ophthalmic composition from an LDPE container having an internal surface to an eye of the subject, wherein the LDPE comprises a density of about 0.925 g/mL to about 0.927 g/mL (e.g., about 0.926 g/mL to about 0.927 g/mL) and a melting point of about 114° C., wherein the LDPE container is prepared by a method of manufacture described herein, wherein the instillation commences up to two years (e.g., less than 730 days, e.g., less than 365 days) from manufacture of the LDPE container, wherein the topical ophthalmic composition is in contact with at least a portion of the internal surface, and wherein the topical ophthalmic composition comprises: about 0.0014% w/v to about 0.004% w/v acoltremon; about 0.14% w/v hypromellose; about 3.0% w/v polyoxyl 35 castor oil; about 0.78% w/v sodium dihydrogen phosphate dihydrate; about 0.55% w/v NaCl; a pH of about 7; and purified water. In some embodiments, the method of manufacture includes methods for manufacture of a topical ophthalmic composition herein, wherein the LDPE container is prepared by a blow-fill-seal process including a blow step to shape at least a portion of the LDPE container and a fill step to fill the portion of the LDPE container with a volume of the topical ophthalmic composition, and the LDPE container is maintained at a temperature of not more than 30° C. for at least 365 consecutive days (e.g., at least 2 years, e.g., at least 730 days) immediately subsequent to the seal step of the blow-fill-seal process.

In some embodiments, provided herein are methods of treating one or more signs or symptoms of dry eye disease in a subject in need thereof, comprising instillation of a topical ophthalmic composition from an LDPE container having an internal surface to an eye of the subject, wherein the LDPE comprises a density of about 0.925 g/mL to about 0.927 g/mL (e.g., about 0.926 g/mL to about 0.927 g/mL) and a melt mass-flow rate (ISO 1133:2005 or ASTM D1238-10) of about 0.30 g/(10-min) to about 0.40 g/(10-min) (e.g., about 0.30 g/(10-min) or about 0.33 g/(10-min) to about 0.40 g/(10-min)), wherein the LDPE container is prepared by a method of manufacture described herein, wherein the instillation commences up to two years (e.g., less than 730 days, e.g., less than 365 days) from manufacture of the LDPE container, wherein the topical ophthalmic composition is in contact with at least a portion of the internal surface, and wherein the topical ophthalmic composition comprises: about 0.0014% w/v to about 0.004% w/v acoltremon; about 0.14% w/v hypromellose; about 3.0% w/v polyoxyl 35 castor oil; about 0.78% w/v sodium dihydrogen phosphate dihydrate; about 0.55% w/v NaCl; a pH of about 7; and purified water. In some embodiments, the method of manufacture includes methods for manufacture of a topical ophthalmic composition herein, wherein the LDPE container is prepared by a blow-fill-seal process including a blow step to shape at least a portion of the LDPE container and a fill step to fill the portion of the LDPE container with a volume of the topical ophthalmic composition, and the LDPE container is maintained at a temperature of not more than 30° C. for at least 365 consecutive days (e.g., at least 2 years, e.g., at least 730 days) immediately subsequent to the seal step of the blow-fill-seal process.

In some embodiments, the physical properties referred to herein may be quantified according to pertinent established methodologies, e.g., ASTM or ISO methodologies, such as those referred to herein.

### EXAMPLES

The present disclosure may be better understood by reference to the following non-limiting examples, which are provided as exemplary of the present disclosure. The following examples are presented to more fully illustrate embodiments of the present disclosure. However, they are in no way a limitation of the teachings or disclosure as described herein.

### Example 1. Acoltremon Compositions

TABLE 5
Acoltremon compositions.

| Component | Composition 1 | | Composition 2 | |
|---|---|---|---|---|
| Acoltremon | 0.003% | w/v | 0.0014% | w/v |
| Hypromellose | 0.14% | w/v | 0.14% | w/v |
| Polyoxyl 35 castor oil | 3.0% | w/v | 3.0% | w/v |
| Sodium dihydrogen | 0.78% | w/v | 0.78% | w/v |
| phosphate dihydrate | | | | |
| NaCl | 0.55% | w/v | 0.55% | w/v |

| | | |
|---|---|---|
| pH | 7 | 7 |
| Water | qs to final volume | qs to final volume |
| | (e.g., 0.25-0.4 mL fill | (e.g., 0.25-0.4 mL fill |
| | volume) | volume) |

### Example 2. LDPE-Contained Acoltremon Compositions

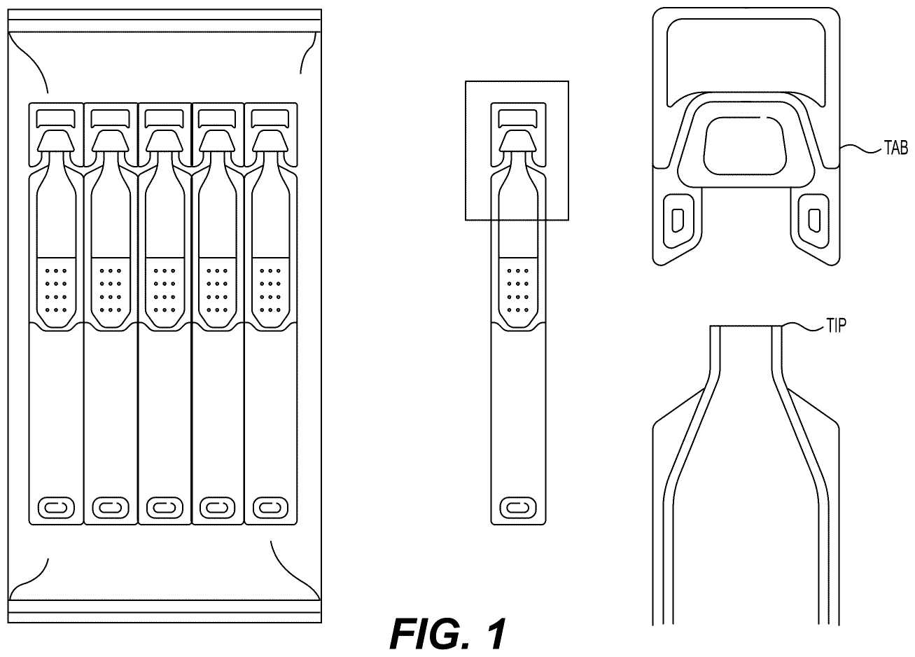

Composition 1 and Composition 2 (Table 5) were prepared in BFS single-use vials using either Sabic PCG00 LDPE or Purell PE3020D LDPE. Each vial contained about 0.25 to about 0.4 mL of Composition 1 or Composition 2. Examples of BFS single-use vials are depicted in FIG. 1.

### Example 3. Clinical Studies

The therapeutic utility of acoltremon is addressed in U.S. Ser. No. 11/850,221B2, which is incorporated herein by reference. The therapeutic utility of acoltremon is further described below.

Studies in animals suggest that acoltremon, the active substance in Composition 1 and Composition 2, is a potent and selective agonist of transient receptor potential melastatin 8 (TRPM8) thermoreceptors. The efficacy of Composition 1 or Composition 2 in DED is believed to be the result of TRPM8 activation of the trigeminal parasympathetic pathway leading to stimulation of basal tear production and increased blink rate. The exact mechanism of action for Composition 1 and Composition 2 in DED is unknown at this time.

In a subset of dry eye disease patients enrolled in a Phase 3 trial and exposed to Composition 1 (n=25), the pre-dose (trough) and post-dose plasma concentrations were assessed at 15 minutes, 30 minutes, 1 hour, 4 hours, and 8 hours after Composition 1 administration (1 drop twice daily). A total of three (3) of the 25 patients (12.0%) had plasma concentrations above 20 pg/mL (lower limit of quantification of the assay). The range of plasma concentrations was 22.8 pg/mL (lowest) to 213 pg/mL (highest). Plasma Cₘₐₓ and other parameters could not be calculated as most samples were below the lower limit of quantification.

The efficacy of Composition 1 for the treatment of the signs and symptoms of DED was supported by two randomized, multi-center, double-masked, vehicle-controlled studies (COMET-2 [NCT-05285644] and COMET-3 [NCT-05360966]) enrolling a total of 931 patients (462 of which received Composition 1).

Patients were randomized to Composition 1 or vehicle (placebo) in a 1:1 ratio and dosed twice a day for 90 days (e.g., instill one drop twice daily in each eye (approximately 12 hours apart); if one dose is missed, treatment continues with the next dose). The BFS single use vials were stored within a temperature range of from 2-25° C. during the study. Use of artificial tears was not allowed during the studies. The mean age was 61 years (range, 30-93 years). The majority of patients were female (74.8%). Enrollment criteria included signs (i.e., corneal fluorescein staining score [2-15] and anesthetized Schirmer tear test [2-9 mm]) and symptoms (i.e., SANDE Score [≥50] and Ocular Discomfort Score [≥50]) of DED.

### Efficacy on Signs of Dry Eye Disease

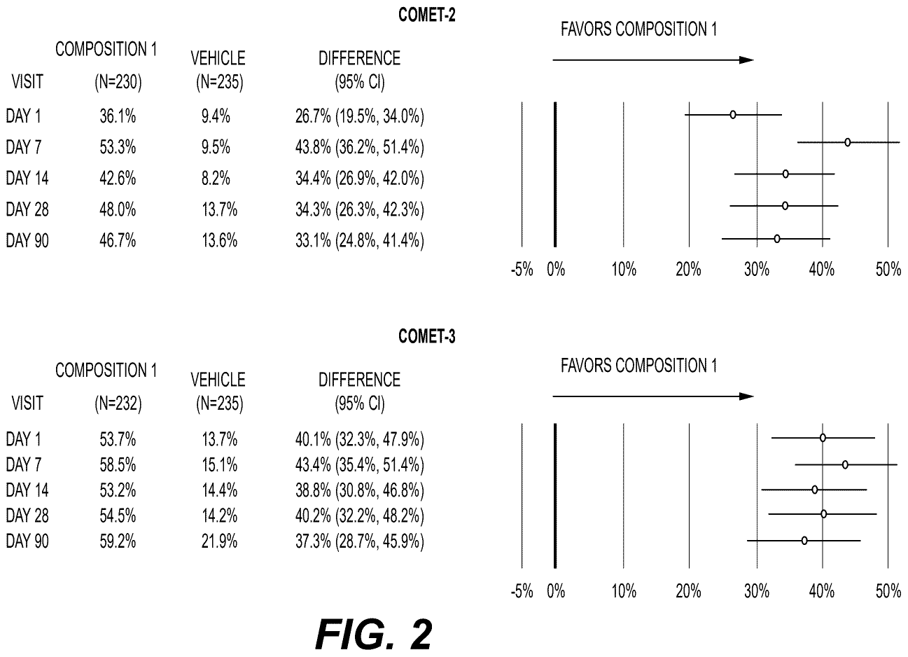

Tear film production was measured by unanesthetized Schirmer tear test assessed using a Schirmer strip (0-35 mm). The average baseline unanesthetized Schirmer scores for Composition 1 and vehicle treated patients was 6.2 mm and 5.9 mm in the COMET-2 study, respectively, and 6.8 mm and 6.4 mm, respectively, in the COMET-3 study. Of the patients treated at Day 14 with Composition 1, 42.6% achieved ≥10 mm increase in Schirmer score from baseline in the COMET-2 study and 53.2% achieved ≥10 mm increase in Schirmer score from baseline at Day 14 in the COMET-3 study, compared to 8.2% and 14.4% of vehicle-treated patients in the COMET-2 study and the COMET-3 study, respectively (see Table 6). Overall, patients treated with Composition 1 had a greater increase in basal tear production at all timepoints measured from Day 1 to Day 90, p<0.0001 (see FIG. 2). Consistent with increased basal tear production, a reduction in ocular surface staining was observed at Day 7 through Day 90 (p<0.005).

TABLE 6
Percent of patients achieving ≥10 mm improvement from baseline
in Schirmer score at day 14 in patients with dry eye disease.
Tear Production

| | COMET-2 | | COMET-3 | |
|---|---|---|---|---|
| | Composition 1 | Vehicle | Composition 1 | Vehicle |
| | N = 230 | N = 235 | N = 232 | N = 234 |
| ≥10 mm increase in tear production | 42.6% | 8.2% | 53.2% | 14.4% |
| at Day 14 | | | | |

| | | |
|---|---|---|
| Difference (95% CI) | 34.4% (26.9, 42.0) | 38.8% (30.8, 46.8) |
| P-value versus vehicle | <0.0001 | <0.0001 |

### Efficacy on Symptoms of Dry Eye Disease

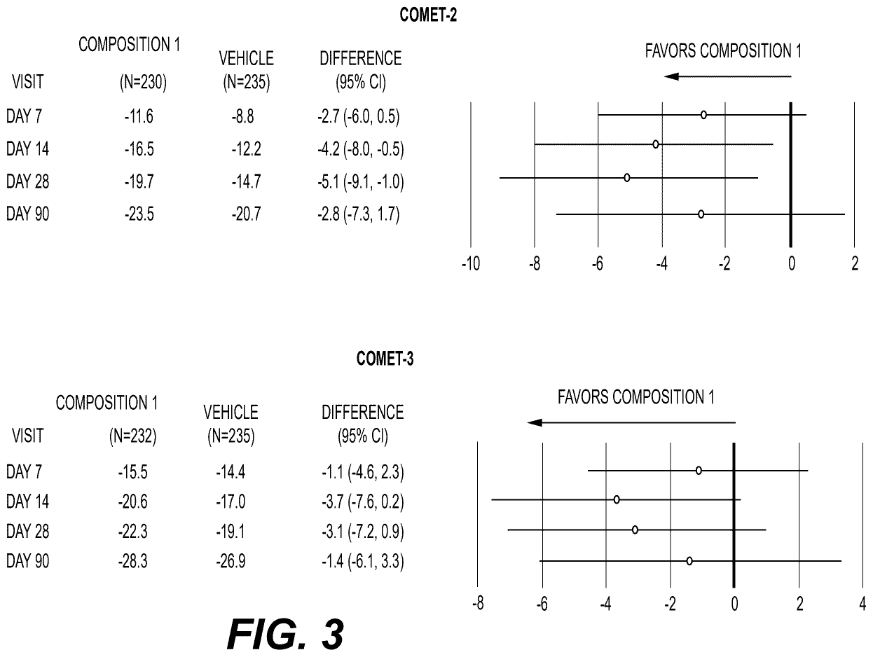

Symptoms of DED were rated by patients using the SANDE (Symptom Assessment iN Dry Eye) Questionnaire which is composed of two visual analogue scales measuring the frequency (0=rarely, 100=all the time) and severity (0=very mild, 100=very severe) of ocular dryness and/or irritation. The average SANDE score at baseline for Composition 1 and vehicle treated patients was 73 and 73, respectively, in the COMET-2 study and 73 and 74, respectively, in the COMET-3 study. A greater reduction in favor of Composition 1 treated patients compared to vehicle treated patients was observed in both COMET-2 (−19.7 vs −14.7) and COMET-3 (−22.3 vs −19.1) (see Table 7). Overall, patients treated with Composition 1 had a greater reduction in symptoms at all timepoints measured compared to vehicle (from Day 7 to Day 90) (see FIG. 3).

TABLE 7
Mean change from baseline in SANDE score at day 28 in patients with dry eye disease.
Tear Production

| | COMET-2 | | COMET-3 | |
|---|---|---|---|---|
| | Composition 1 | Vehicle | Composition 1 | Vehicle |
| | N = 230 | N = 235 | N = 232 | N = 234 |
| Mean Δ from baseline at Day 28 | −19.7 | −14.7 | −22.3 | −19.1 |

| | | |
|---|---|---|
| Difference (95% CI) | −5.1 (−9.1, −1.0) | −3.1 (−7.2, 0.9) |
| P-value versus vehicle | 0.0138 | 0.1321 |

### Example 4. Acoltremon Sorption to Container Inner Surface

As described in U.S. Ser. No. 11/850,221B2, an inverse relationship was observed between the crystallinity of a polyolefin and the amount of acoltremon absorbed into the polyolefin when the acoltremon composition is stored in contact with the polyolefin at elevated temperatures (e.g. 40° C.). Table 8, reproduced here from U.S. Ser. No. 11/850,221B2, shows that polyolefins with high melting points, such as HDPE and PP, yielded the lowest percent loss in acoltremon, while polyolefins with low melting points, such as LDPE 5, showed the greatest loss of acoltremon.

TABLE 8
Melting temperatures of various polyolefins correlated with the
percent loss of acoltremon concentration stored in the polyolefin
container (reproduced in part from U.S. Pat. No. 11,850,221B2;
rows corresponding to SABIC PCG00 and DOW450E added).

| | | | Percent Loss in |
|---|---|---|---|
| | | | acoltremon |
| | | | Concentration |
| | Melting Peak | | After Storage in |
| | Temperature | Density of | Polyolefin |
| | of Polyolefin | Polyolefin | Container for 2 |
| Polyolefin Container Material | by DSC (° C.) | (g/cm³) | Weeks at 40° C. |
| High Density Polyethylene (HDPE) | 127.4 | 0.950 | 1.3 |
| Polypropylene (PP) | 149.1 | 0.902 | 1.3 |
| Low Density Polyethylene (LDPE 1) | 118.2 | 0.933 | 2.8 |
| Low Density Polyethylene (LDPE 2) | 116.0 | 0.930 | 2.8 |
| Low Density Polyethylene (LDPE 3) | 114.4 | 0.928 | 3.3 |
| Low Density Polyethylene (LDPE 4) | 113.1 | 0.927 | 4.0 |
| SABIC PCG00 | 112.2 | 0.925 | |
| DOW450E | 111.9 | 0.923 | |
| Low Density Polyethylene (LDPE 5) | 107.8 | 0.919 | 4.5 |

LDPE 1, LDPE 2, LDPE 3, LDPE 4, and LDPE 5 in Table 8 correspond to LDPEs from different suppliers. Melting point values of the polyolefins were experimentally determined by differential scanning calorimetry (DSC). The densities of the polyolefins were as provided by the manufacturer's product data sheet.

Composition 1 was prepared in BFS single-use vials using LDPE 1, LDPE 2, LDPE 3, LDPE 4, LDPE 5, DOW 450E, and SABIC PCG00, and a 193° C. melt temperature for blown film. Each vial contained 5 mL (10 mL internal volume of blown vial), 0.5 mL (1 mL internal volume of blown vial), 0.4 mL (1 mL internal volume of blown vial), or 0.25 mL (1 mL internal volume of blown vial) of Composition 1 (Table 9). When the BFS units were stored at 25° C., an approximation of a temperature range in which a subject might maintain the sample during its usage without refrigeration, the acoltremon content decreased as depicted in Table 9. Acoltremon concentration beyond approved limits (e.g., below 90.0% of the label active concentration) is unacceptable. The decrease in acoltremon content (e.g., as demonstrated in FIG. 8) was not predictable based on the data from LDPEs 1-5 for lack of following a trend of acoltremon loss determinable based on storage temperature and fill volume. These data in combination with data depicted in FIG. 8 demonstrate the surprising capacity of SABIC PCG) to mitigate loss of acoltremon concentration upon storage at approximately room temperature (e.g., 25° C.), which could not have been predicted by the observations of, e.g., LDPE 5, herein or in U.S. Ser. No. 11/850,22132 (e.g., Table 12 therein).

TABLE 9
Acoltremon content (% label claim) stored in various polyolefin containers.

| | Melting | | 10 mL | 10 mL | 1 mL | 1 mL | 1 mL |
|---|---|---|---|---|---|---|---|
| | Peak | | BFS | BFS | BFS | BFS | BFS |
| | Temp. | | Container | Container | Container | Container | Container |
| | of BFS | | (5 mL | (5 mL | (0.45 mL | (0.4 mL | (0.25 mL) |
| BFS | Material | Density of | fill) for | fill) for | fill) for | fill) for | for |
| Container | by DSC^{†} | Polyolefin | 2 weeks | 1 month | 1 month | 1 month | 1 month |
| Material | (° C.) | (g/cm³) | at 40° C. | at 25° C. | at 25° C. | at 25° C. | at 25° C. |
| LDPE 1 | 118.2 | 0.933 | 98% | 100% | | | |
| LDPE 2 | 116.0 | 0.930 | 98% | 99% | | | |
| LDPE 3 | 114.4 | 0.928 | 98% | 99% | | | |
| LDPE 4 | 113.1 | 0.927 | 97% | 99% | | | |
| SABIC | 112.2 | 0.925 | | | | 97%^{‡} | 97%^{‡} |
| PCG00 | | | | | | | |
| LDPE 5 | 107.8 | 0.919 | 96% | 99% | 89% | | |
| DOW | 111.9 | 0.923 | | | 91% | | |
| 450E | | | | | | | |

^{†}After BFS manufacture.
^{‡}Estimated from FIG. 8.

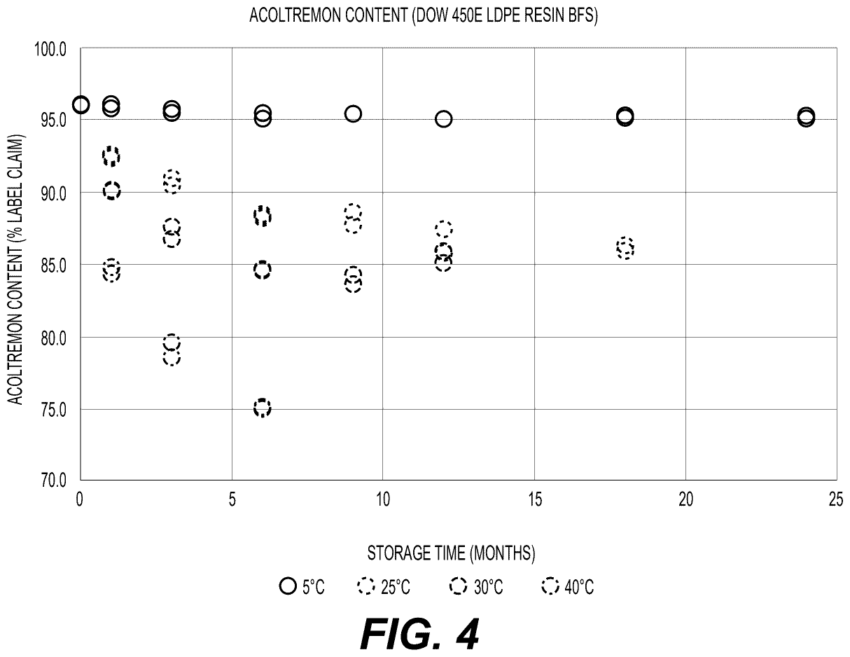

Composition 1 was prepared in BFS single-use vials using DOW(TM) LDPE 450E resin (0.923 g/cm³ density and 2.0 g/10 min MFR (ASTM D1238)), and a 193° C. melt temperature for blown film. Each vial contained about 0.25 to about 0.4 mL of Composition 1. When the BFS units were stored at temperatures above 5° C. (long term stability at 5° C., 25° C./60% RH, 30° C./65% RH and 40° C./75% RH for a period of up to 24 months or longer), the acoltremon content decreased significantly. Increasing storage temperature and storage time resulted in problematically increasing losses of acoltremon to sorption (see FIG. 4).

Composition 1 was prepared in BFS single-use vials using Purell 1840H LDPE resin (0.919 g/cm³ density (ASTM D1505), 1.5 g/10 min MFR (ASTM D1238), and 108° C. peak melt temperature), and a 193° C. melt temperature for blown film. Each vial contained about 0.25 to about 0.4 mL of Composition 1. When the BFS units were stored at temperatures above 5° C. (long term stability at 5° C., 25° C./60% RH, 30° C./65% RH and 40° C./75% RH for a period of 24 months), the acoltremon content decreased significantly. Increasing storage temperature and storage time resulted in problematically increasing losses of acoltremon to sorption (see FIG. 5).

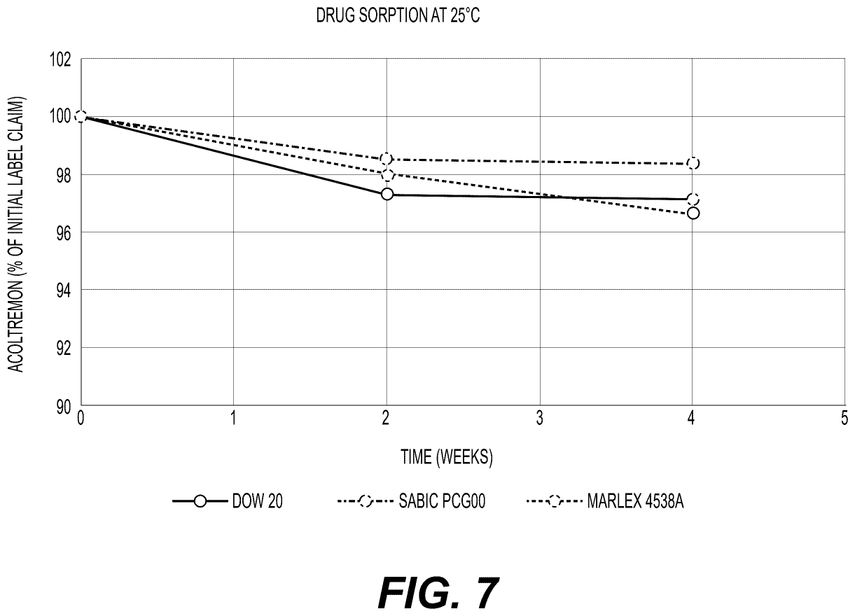

Composition 1 was prepared in BFS single-use vials using Sabic PCG00 (0.925-0.926 g/cm³ density and 0.33-0.4 g/10 min MFR), DOW 20 (0.920 g/cm³ density (ISO 1183) and 1.9 g/10 min MFR (ASTM D1238)), and Marlex 4538A (0.925 g/cm³ density (ASTM D1505) and 2.0 g/10 min MFR (ASTM D1238)) LDPE resins. Each vial contained about 0.25 or about 0.4 mL of Composition 1. The BFS units were stored at temperatures above 5° C. (long term stability at 25° C./60% RH and 30° C./65% RH for a period of 4 weeks). FIG. 6 and FIG. 7 show the sorption trends of acoltremon to BFS units made of the three resins. The extent of drug loss increased as the storage temperature was increased. At all temperatures, the Sabic PCG00 resin performed the best, showing the lowest potential to adsorb/absorb acoltremon.

Composition 1 was prepared in BFS single-use vials using Sabic PCG00 and Purell 3020D LDPE resins. Each vial contained about 0.25 or about 0.4 mL of Composition 1. The BFS units were stored at 25° C./60% RH for a period of 32 weeks). FIG. 8 shows the sorption trends of acoltremon to BFS units made of the two resins. The Sabic PCG00 resin performed slightly better than the Purell 3020D resin when the fill volume was 0.25 mL, but the two resins performed equivalently with a fill volume of 0.4 mL. For both resins, the larger fill volume of 0.4 mL performed significantly better than the 0.25 mL fill volume. After 32 weeks storage at 25° C./60% RH, the 0.4 mL fill volume showed at least a 4% higher label claim for acoltremon compared to the 0.25 mL fill volume. This result clearly indicates that maintaining a low surface area to volume ratio for the product in the BFS is another factor in minimizing acoltremon adsorptive/absorptive losses.

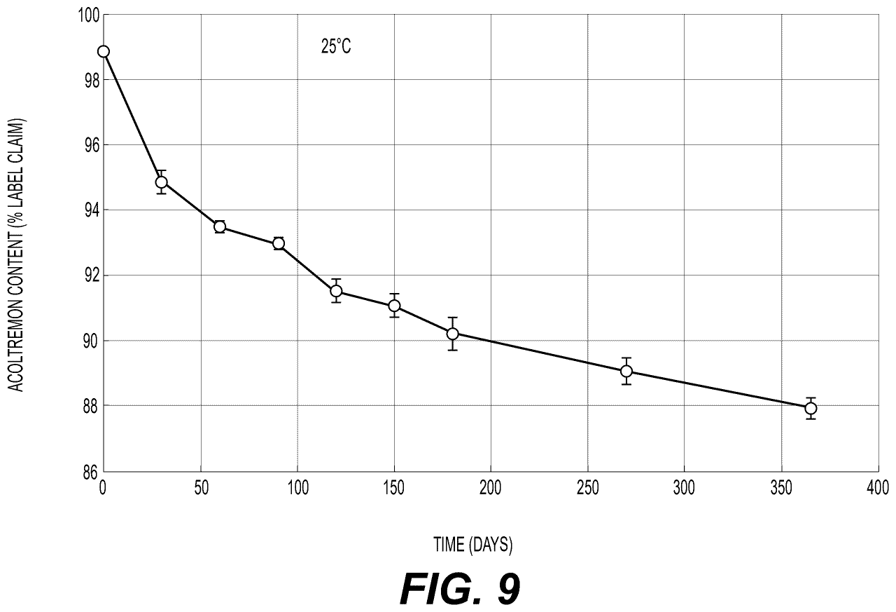

Composition 1 was prepared in BFS single-use vials using Sabic PCG00 LDPE resin. Each vial contained about 0.4 mL of Composition 1. The BFS units were sealed in aluminum pouches (5 units/pouch) and stored at 25° C./60% RH for a period of 32 weeks). The results shown in FIG. 9 demonstrate that the product could be stored at room temperature for up to 30 days during the use period with the patient.

These data taken as a whole, it was surprisingly found that the LDPE-contained acoltremon compositions herein provide improvements on a variety of fronts, including commercial manufacture or cost, ease of transport or storage, patient compliance, and consistency of label strength of acoltremon during storage.

### Example 5. Acoltremon Sorption to Container Inner Surface

The LDPE sorption of acoltremon was assessed in 1 mL BFS units made of Sabic PCG00, Purell 3020D, or Purell 1840H LDPE polymers filled with 0.4 mL (Sabic PCG00 and Purell 3020D BFS units) or 0.45 mL (Purell 1840H BFS units) of drug product (0.003% w/v acoltremon; Composition 1). The filled units were sealed in foil bags and placed on stability at 25° C./40% RH. Duplicate samples were tested by HPLC for acoltremon content at T₀ and pre-determined intervals up to 32 weeks.

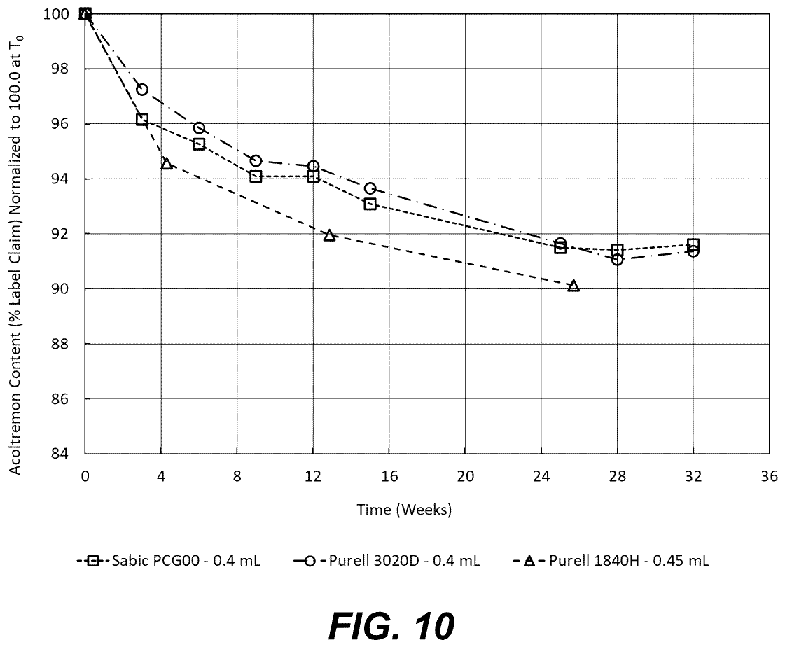

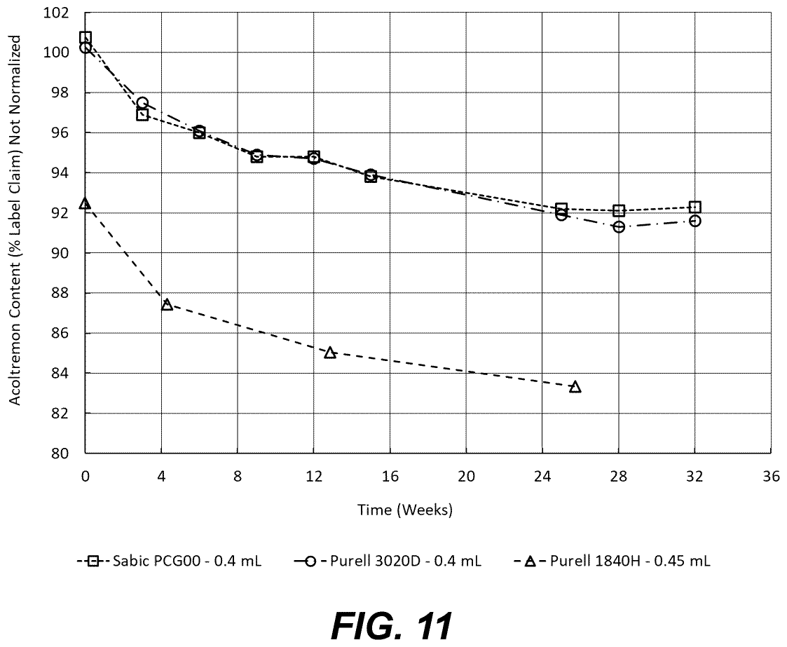

Results are depicted in FIG. 10 (normalized to 100.0% at T₀) and FIG. 11 (not normalized). BFS units of Sabic PCG00 or Purell 3020D were observed to sorb less acoltremon (as a sorption rate or as total sorption) than BFS units of Purell 1840H (LDPE 5) when stored at 25° C.

BFS units with Composition 2 can be similarly assessed.

### Example 6. Acoltremon Sorption to Polyolefin Beads

The LDPE sorption of acoltremon to polyolefin resin beads was assessed. Approximately 9 g of resin beads were weighed into a scintillation vial, and the beads were immersed in 7 mL of drug product (0.003% w/v acoltremon; Composition 1). Resins used were Marlex 4538A, Purell 3020D, Sabic PCG00, Dupont 20, Dow 450E, Dow Health 690, Dupont 20-6064, Bormed LE-6600PH, and Purell 1840H. The bead volume to formulation volume ratio was 1.4 for all samples. The volume of resin beads was calculated by dividing the mass (g) of the beads by the resin density (g/mL) taken from the resin manufacturer's Technical Data Sheet. The vials containing formulation and resin beads were capped tightly, sealed with parafilm, and stored at 40° C., along with a control vial containing formulation with no resin beads. The vials containing resin beads and the control were sampled at pre-determined time points and analyzed for acoltremon content by HPLC. Drug product stored in glass at 5° C. served as a surrogate To sample.

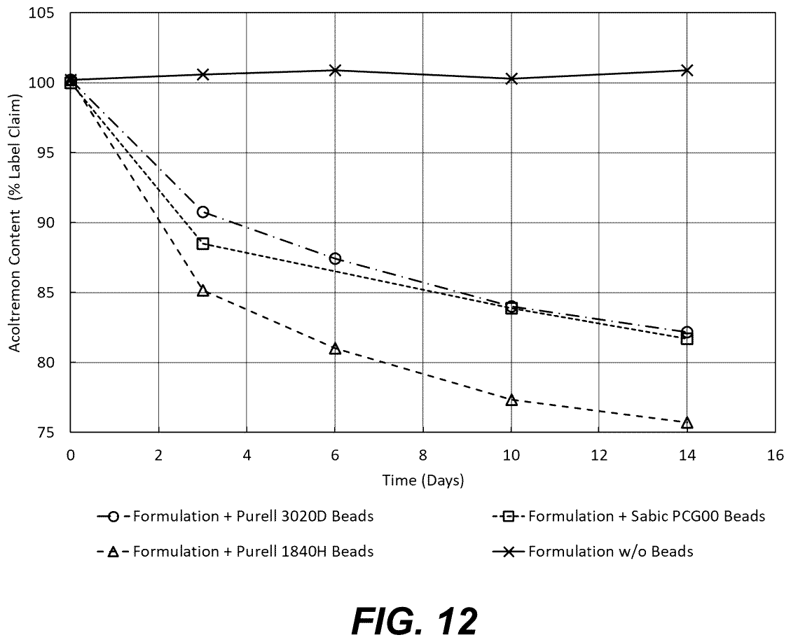

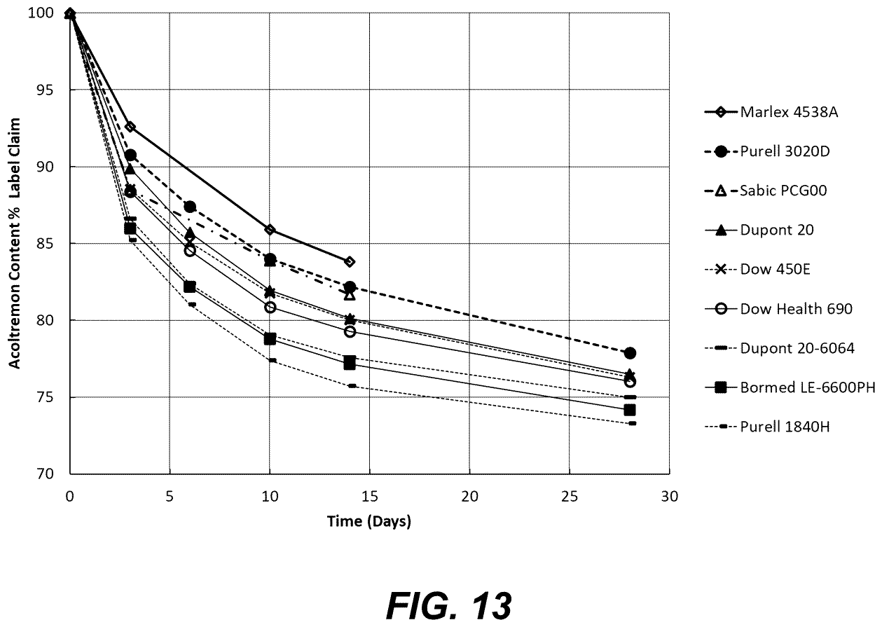

Results are depicted in FIG. 12 (not normalized) and FIG. 13 (normalized to 100.0% at To).

Resins in the presence of Composition 2 can be similarly assessed.

### Example 7. Acoltremon Sorption to Container Inner Surface

The LDPE sorption of acoltremon was assessed in 1 mL BFS units made of Sabic PCG00 or Purell 1840H LDPE polymers filled with 0.4 mL (Sabic PCG00 and Purell 3020D BFS units) or 0.45 mL (Purell 1840H BFS units) of drug product (0.003% w/v acoltremon; Composition 1). The filled units were sealed in foil pouches and placed on stability at 25° C./40% RH. Duplicate samples were tested by HPLC for acoltremon content at T₀ and pre-determined intervals up to 1 month.

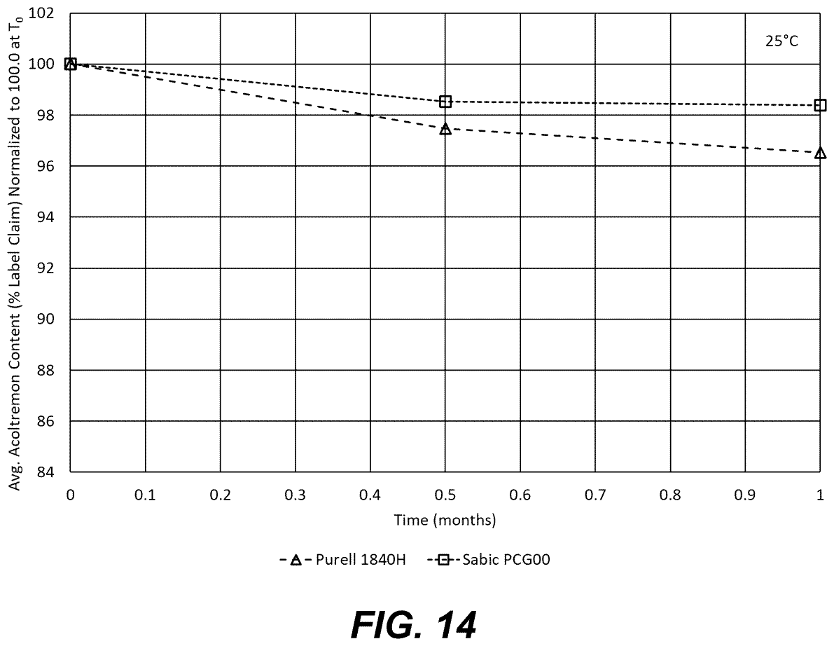

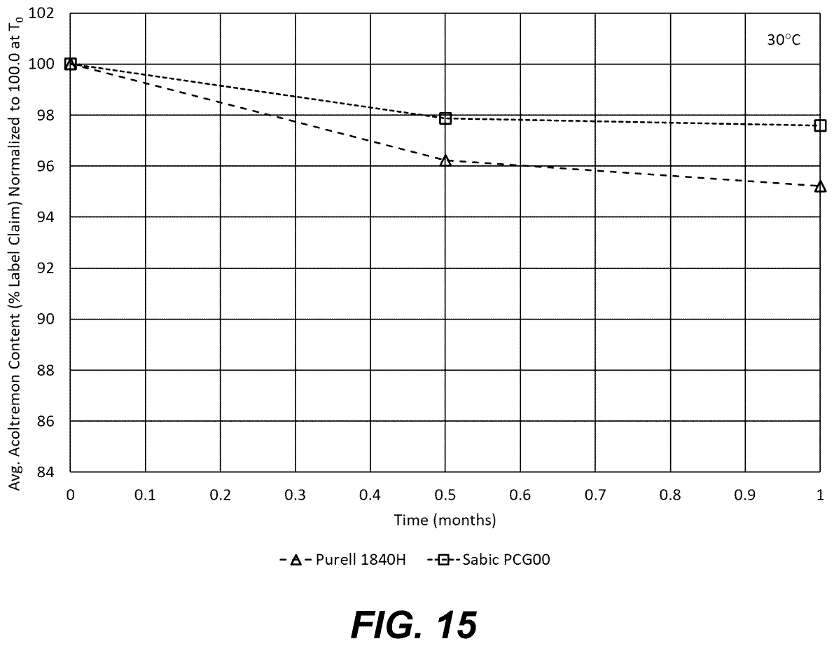

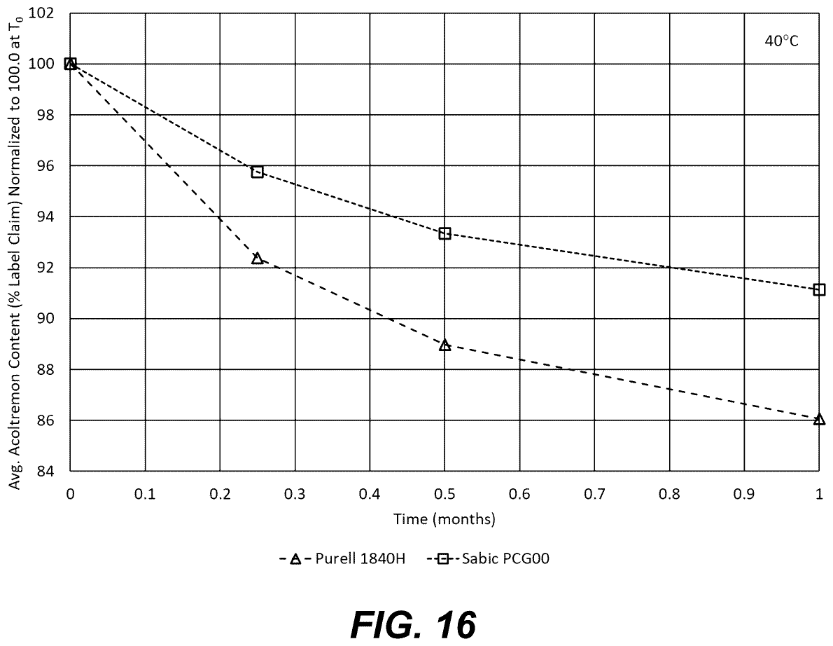

Results are depicted in FIG. 14 (25° C.; normalized to 100.0% at T₀), FIG. 15 (30° C.; normalized to 100.0% at T₀), and FIG. 16 (40° C.; normalized to 100.0% at T₀). BFS units of Sabic PCG00 were observed to sorb nearly 10% of the acoltremon content when stored at 40° C., and BFS units of Purell 1840H were observed to sorb about 14% of the acoltremon content when stored at 40° C. BFS units of Purell 1840H with Composition 1 would have to be filled at a concentration higher than label claim in order to meet FDA standards for potency on storage, particularly if a heat cure step were incorporated into the BFS manufacture process as proposed by U.S. Ser. No. 11/850,221, which would lead to an increased cost of manufacture at least with respect to cost of acoltremon. At 0.5 months (about 2 weeks) of storage at 40° C., BFS units of Sabic PCG00 were observed to sorb 6.7% acoltremon content. However, at one month of storage at either 25° C. or 30° C. (without a 40° C. heat cure step) BFS units of Sabic PCG00 were observed to sorb only 1.6% or 2.4%, respectively, acoltremon content, whereas BFS units of Purell 1840H were observed to sorb 3.5% or 4.8%, respectively, acoltremon content—at least twice that of the Sabic PCG00 BFS units under the same conditions. When stored at 30° C., BFS units of Sabic PCG00 performed better in terms of absolute acoltremon sorption than BFS units of Purell 1840H stored at 25° C.

BFS units with Composition 2 can be similarly assessed.

### Example 8. Acoltremon Sorption to Container Inner Surface

The LDPE sorption of acoltremon was assessed in 10 mL BFS units made (unfilled) of various LDPE polymers. The empty BFS vials were cut open, filled with 5 mL of drug product (0.003% w/v acoltremon; Composition 1), and re-sealed by softening the vial neck with a heat gun and sealing it with pliers. The filled units were sealed in foil pouches and placed on stability at 25° C./40% RH, 30° C./35% RH, 35° C./ambient RH, and 40° C./ambient RH. Scintillation vials containing 5 mL of drug product were also placed on stability as controls. Duplicate samples were tested by HPLC for acoltremon content at T₀ and pre-determined intervals.

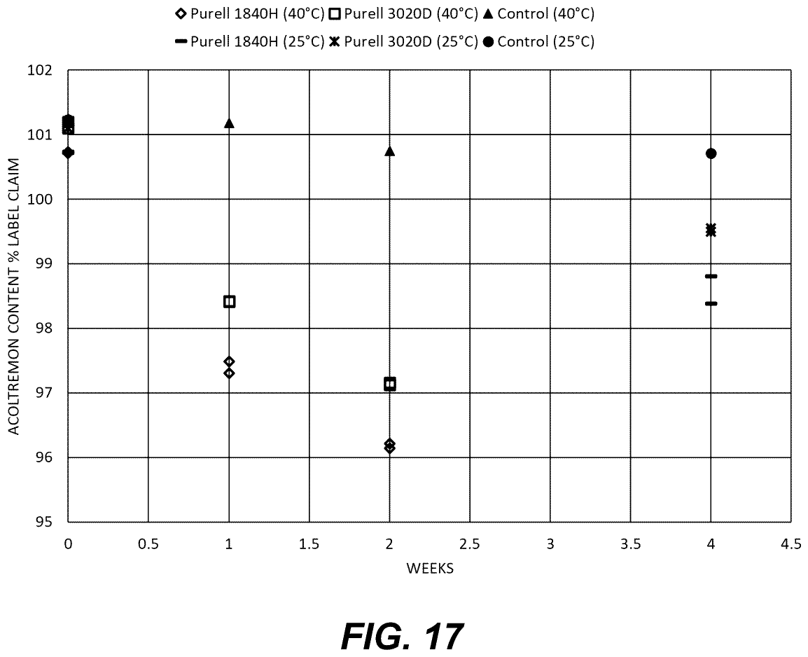

Results are depicted in FIG. 17. BFS units of Purell 3020D were observed to sorb less acoltremon (as a sorption rate or as total sorption) than BFS units of Purell 1840H (LDPE 5) at 25° C. for 4 weeks and at 40° C. for 2 weeks.

BFS units with Composition 2 can be similarly assessed.

### Example 9. Differential Scanning Calorimetry (DSC)

BFS units were sampled by taking a 4 mm punch of the polyolefin (e.g., LDPE) BFS unit. DSC was performed on punch samples. DSC was also performed on commercially supplied resin beads. DSC conditions included: T zero pan; ~8 mg resin sample/~10 mg disc; Heat/Cool/Heat:10° C./min, Quench cool, 10° C./min. Results are described in Table 9, above.

### Example 10. High Performance Liquid Chromatography (HPLC)

HPLC as described in the examples herein was generally performed according to the protocol described in Table 10. External standards of acoltremon were made at 0.03 mg/mL in (80/20) methanol/water. Samples were quantitated against the external standards.

TABLE 10
HPLC protocol.

| | |
|---|---|
| Analytical | Chiralcel OD-3R analytical column, 15 cm × 4.6 mm id, 3 μm particles, with |
| Column | Chiralcel OD-3R guard cartridge, 10 mm × 4.0 mm id, 3 μm particles |
| Column | 40° C. |

Temperature

| | |
|---|---|
| Autosampler | Ambient (or set to an appropriate temperature, such as 20° C.) |

| | | |
|---|---|---|
| Temperature | | |
| Injection | Working Standard Solutions | 35 μL |
| Volumes | Sensitivity Check Solution | |
| | Marker Solution | |
| | 0.003% drug product (Composition 1) | |
| | Diluent, injected before Sensitivity Check Solution | |
| | 0.0014% drug product (Composition 2) | 70 μL |
| | Placebo | |
| | Diluent, injected before Placebo | |

| | |
|---|---|
| Flow Rate | 1.0 mL/min |
| Mobile Phase A | Deionized Water |
| Mobile Phase B | Methanol |
| Mobile Phase C | Acetonitrile |
| Needle Rinse | Methanol |
| Seal Wash | (80/20) Water/Methanol |

| | Time (minutes) | % Mobile Phase A | % Mobile Phase B | % Mobile Phase C |
|---|---|---|---|---|
| Pump Gradient | 0 | 60 | 38 | 2 |
| Pattern | 28 | 10 | 88 | 2 |
| | 29 | 10 | 0 | 90 |
| | 38 | 10 | 0 | 90 |
| | 39 | 60 | 38 | 2 |
| | 45 | 60 | 38 | 2 |

| | |
|---|---|
| Total Run Time | 45 minutes |
| Detection | From 0-8 minutes, 232 nm |
| Wavelength | At 8 minutes, switch the wavelength to 249 nm |
| | For identification testing, use 200-400 nm for photodiode array UV |
| | spectra |

The contents of all references (including literature references, issued patents, published patent applications, and co-pending patent applications) cited throughout this application are hereby expressly incorporated herein by reference in their entireties. Unless otherwise defined, all technical and scientific terms used herein are accorded the meaning commonly known to one with ordinary skill in the art.

1. A topical ophthalmic composition, wherein the topical ophthalmic composition is contained in a low-density polyethylene (LDPE) container, the LDPE container comprises an internal surface, the topical ophthalmic composition is in contact with at least a portion of the internal surface, the LDPE comprises two or more of a melt mass-flow rate of 0.30 to 0.40 grams per 10 minutes (g/(10-min)), a density of 0.925 g/mL to 0.927 g/mL, or a peak melting point of 114° C., and the topical ophthalmic composition comprises:
0.0014% to 0.004% w/v acoltremon;
3.0±5% w/v polyoxyl 35 castor oil;
0.78±5% w/v sodium dihydrogen phosphate dihydrate;
0.55±5% w/v NaCl;
a pH of 7; and
purified water.

2. The topical ophthalmic composition of claim 1, wherein the LDPE comprises a melt mass-flow rate of 0.30 to 0.40 g/(10-min), a density of 0.925 g/mL to 0.927 g/mL, and a peak melting point of 114° C..

3. The topical ophthalmic composition of claim 1, wherein the topical ophthalmic composition further comprises at least one of:
a viscosity of not more than 5 cP;
an osmolality of 280 to 330 mOsm/kg; or
a density of 1.011 g/mL at a temperature of 20° C. to 25° C.

4. The topical ophthalmic composition of claim 1, wherein the topical ophthalmic composition has a volume of 0.25±0.05 mL, 0.3±0.05 mL, 0.4±0.05 mL, 0.5±0.05 mL, 0.6±0.05 mL, 0.7±0.05 mL, or 1±0.05 mL.

5-7. (canceled)

8. The topical ophthalmic composition of claim 1, wherein the topical ophthalmic composition comprises:
0.0014±5% w/v acoltremon, 0.003±5% w/v acoltremon, or 0.004±5% w/v acoltremon; and
a volume of 0.25±0.05 mL or 0.4±0.1 mL.

9. The topical ophthalmic composition of claim 1, wherein the LDPE container is a blow-fill-seal container or sealed in a foil pouch, or a combination thereof.

10. The topical ophthalmic composition of claim 1, wherein the LDPE comprises:
a density of 0.925-0.926 g/mL, a melting point of 114° C., and a melt mass-flow rate of 0.33-0.40 g/(10-min); or
a density of about 0.927 g/mL, a melting point of about 114° C., and a melt mass-flow rate of about 0.30 g/(10-min).

11. A method of treating one or more signs or symptoms of dry eye disease in a subject in need thereof, comprising instillation of a dosage amount of the topical ophthalmic composition of claim 1 to an eye of the subject.

12. The method of claim 11, wherein the dosage amount has a volume of 0.025±0.005 mL, 0.03±0.005 mL, 0.04±0.005 mL, 0.05±0.005 mL, 0.06±0.005 mL, or 0.07±0.005 mL.

13. The method of claim 11, wherein the topical ophthalmic composition has not been exposed to a temperature above 30° C. subsequent to its being contained in the LDPE container.

14. (canceled)

15. The method of claim 11, wherein the dosage amount has a volume of 0.025 mL to 0.04 mL.

16. The method of claim 11, wherein the instillation is once or twice daily to each eye of the subject.

17-19. (canceled)

20. A method for manufacture of the topical ophthalmic composition of claim 1, wherein the LDPE container is prepared by a blow-fill-seal process including a blow step to shape at least a portion of the LDPE container and a fill step to fill the portion of the LDPE container with a volume of the topical ophthalmic composition, and the LDPE container is maintained at a temperature of not more than 30° C. for at least 15 weeks immediately subsequent to the seal step of the blow-fill-seal process.

21. A method of treating one or more signs or symptoms of dry eye disease in a subject in need thereof, comprising instillation of a topical ophthalmic composition from an LDPE container having an internal surface to an eye of the subject,
wherein the LDPE comprises a density of 0.925 g/mL to 0.927 g/mL and a melting point of 114° C.,
wherein the topical ophthalmic composition is prepared by the method for manufacture of claim 20,
wherein the instillation commences less than 30 weeks from manufacture of the LDPE container;
wherein the topical ophthalmic composition is in contact with at least a portion of the internal surface, and
wherein the topical ophthalmic composition comprises:
0.0014% w/v to 0.004% w/v acoltremon;
0.14±5% w/v hypromellose;
3.0±5% w/v polyoxyl 35 castor oil;
0.78±5% w/v sodium dihydrogen phosphate dihydrate;
0.55±5% w/v NaCl;
a pH of 7; and
purified water.

22. A method of treating one or more signs or symptoms of dry eye disease in a subject in need thereof, comprising instillation of a topical ophthalmic composition from an LDPE container having an internal surface to an eye of the subject,
wherein the LDPE comprises a density of 0.925 g/mL to 0.927 g/mL and a melting point of 114° C.,
wherein the topical ophthalmic composition is prepared by the method for manufacture of claim 20,
wherein the instillation commences between 30 weeks and 3 years from manufacture of the LDPE container;
wherein the topical ophthalmic composition is in contact with at least a portion of the internal surface, and
wherein the topical ophthalmic composition comprises:
0.0014% w/v to 0.004% w/v acoltremon;
0.14±5% w/v hypromellose;
3.0±5% w/v polyoxyl 35 castor oil;
0.78±5% w/v sodium dihydrogen phosphate dihydrate;
0.55±5% w/v NaCl;
a pH of 7; and
purified water.

23. A method of treating one or more signs or symptoms of dry eye disease in a subject in need thereof, comprising instillation of a topical ophthalmic composition from an LDPE container having an internal surface to an eye of the subject,
wherein the LDPE comprises a density of 0.925 g/mL to 0.927 g/mL and a melting point of 114° C.,
wherein the topical ophthalmic composition is prepared by the method for manufacture of claim 20,
wherein the instillation commences at least 3 years from manufacture of the LDPE container;
wherein the topical ophthalmic composition is in contact with at least a portion of the internal surface, and
wherein the topical ophthalmic composition comprises:
0.0014% w/v to 0.004% w/v acoltremon;
0.14±5% w/v hypromellose;
3.0±5% w/v polyoxyl 35 castor oil;
0.78±5% w/v sodium dihydrogen phosphate dihydrate;
0.55±5% w/v NaCl;
a pH of 7; and
purified water.

24. The method of claim 11, wherein the topical ophthalmic composition comprises 0.0014±5% w/v acoltremon, 0.003±5% w/v acoltremon, or 0.004±5% w/v acoltremon.

25. A method of treating an ophthalmic disease or condition in a subject in need thereof, the disease or condition involving tear production, and/or reducing symptoms thereof, the method comprising topically administering to an eye of the subject an administered volume of a topical ophthalmic composition from a single-dose vial,
wherein the single-dose vial is prepared by a method of manufacturing the single-dose vial, the single-dose vial comprising a liquid volume of the topical ophthalmic composition filled in the single-dose vial,
wherein the method of manufacturing the single-dose vial includes a blow-fill-seal (BFS) process, the BFS process comprising blow-forming a low-density polyethylene (LDPE) resin to form a blown vial, filling the blown vial with the liquid volume to form a blown and filled vial, and sealing the blown and filled vial to form the single-dose vial,
wherein the LDPE resin comprises at least two of a melt mass-flow rate of 0.30 to 0.40 grams per 10 minutes (g/(10-min)), a density of 0.925 g/mL to 0.927 g/mL, or a peak melting point of 114° C., and
wherein the topical ophthalmic composition comprises
0.0014% w/v to 0.004% w/v acoltremon,
about 0.14% w/v hypromellose,
about 3.0% w/v polyoxyl 35 castor oil,
about 0.78% w/v sodium dihydrogen Phosphate dihydrate,
about 0.55% w/v NaCl,
a pH of about 7, and
purified water.

26. The topical ophthalmic composition of claim 1, wherein the LDPE container comprises a fillable volume, and a ratio of fillable volume to liquid volume of the topical ophthalmic composition of about 2.2-2.9, about 2.3-2.8, about 2.4-2.7, about 2.4-2.6, about 2.5, or about 1:0.4.

27. The method of claim 11, wherein the LDPE container comprises a fillable volume, and a ratio of fillable volume to liquid volume of the topical ophthalmic composition of about 2.2-2.9, about 2.3-2.8, about 2.4-2.7, about 2.4-2.6, about 2.5, or about 1:0.4.

28. The method of claim 25, wherein the method further comprises, less than one hour after the BFS process, maintaining the single-dose vial at a temperature of not less than 2° C. and not more than about 30° C. for a time period of at least 15 weeks, and
wherein the administered volume is about 25 to about 40 μL.
